(19) United States
(12) Patent Application Publication
(10) Pub. No.: US 2025/0235513 A1
(43) Pub. Date: Jul. 24, 2025

(54) VECTORS AND METHODS FOR THE TREATMENT OF NEURODEGENERATION, DELAYING COGNITIVE DECLINE, AND IMPROVING MEMORY

(71) Applicant: The General Hospital Corporation, Boston, MA (US)

(72) Inventors: Cinzia VICIDOMINI, Cambridge, MA (US); Amar SAHAY, Lexington, MA (US)

(21) Appl. No.: 18/852,568

(22) PCT Filed: Mar. 30, 2023
(86) PCT No.: PCT/US2023/016901

§ 371 Date: Sep. 30, 2024

Related U.S. Application Data
(60) Provisional application No. 63/326,326, filed on Apr. 1, 2022.

(51) Int. Cl. A61K 38/46; A61K 48/00; A61P 25/28; C12N 9/20; C12N 15/86

(52) U.S. Cl. CPC A61K 38/465; A61K 48/005; A61P 25/28; C12N 9/20; C12N 15/86; C12Y 301/01004; C12N 2750/14143

(57) ABSTRACT

The present invention relates to treating neurodegeneration (e.g., a neurodegenerative disorder), enhancing cognitive function, delaying or reducing cognitive decline, enhancing the metabolism, and/or improving memory in patients in need thereof, e.g., patients diagnosed with a neurodegenerative disorder or displaying one or more symptoms of a neurodegenerative disorder (e.g., declining cognitive function) by administering Pla2g2f to a subject or increasing expression of Pla2g2f in a cell of a subject.

### FIELD OF THE INVENTION

The invention relates to the field of therapeutic treatment of neurodegeneration in patients, such as treatment of a neurodegenerative disorder.

### BACKGROUND OF THE INVENTION

Neurodegeneration is the slow and progressive loss of structure or function of neuronal cells in regions of the brain. Pathologies associated with neurodegeneration include neurodegenerative disorders such as Alzheimer's disease, frontotemporal dementia, Parkinson's disease, and Amyotrophic Lateral Sclerosis. Neurodegeneration is also associated with the process of aging and increased neuroinflammation. Symptoms of neurodegeneration include reduced or impaired cognitive function, but can also manifest as impaired motility and balance, disrupted or compromised bladder and bowel function, disrupted sleep, difficulty breathing, declining heart function, and deteriorating speech, among others. Millions of people worldwide are affected by neurodegeneration. Currently, there is an unmet need for better treatments for neurodegeneration.

### SUMMARY OF THE INVENTION

The present disclosure provides compositions and methods that can be used for treating neuroinflammation, neurodegeneration, for example, a neurodegenerative disorder, such as Alzheimer's Disease (AD), frontotemporal dementia (FTD), Parkinson's disease (PD), and Amyotrophic Lateral Sclerosis (ALS), or symptoms of neurodegeneration, including declining cognitive function, and the symptoms of aging. Using the compositions and methods of the disclosure, the expression of Pla2g2f may be increased in a cell in a patient (e.g., a human patient) having a neurodegenerative disorder (e.g., AD, FTD, PD, or ALS) or displaying one or more symptoms of neurodegeneration (e.g., declining cognitive function or increased neuroinflammation). In one embodiment, the patient may be administered a vector (e.g., an expression vector, e.g., an adeno-associated vector (AAV)) encoding phospholipase A2 group IIF (Pla2g2f).

In one aspect, the disclosure provides a method of enhancing cognitive function, delaying cognitive decline, enhancing metabolism, or improving memory in a subject, the method including administering Pla2g2f to the subject. In some embodiments, the Pla2g2f is administered to a cell or a tissue in the subject. In some embodiments, the Pla2g2f is administered systemically. In one embodiment, the method enhances cognitive function in the subject. In another embodiment, the method delays or reduces cognitive decline in the subject. In a further embodiment, the method improves memory in the subject. In one embodiment, the delay or reduction in cognitive decline in the subject is in aging. In another embodiment, the method enhances metabolism in the subject.

In some embodiments, enhancing the metabolism is exhibited by: (a) increased locomotor activity; (b) decreased body weight; (c) decreased food consumption; and/or (d) increased energy expenditure, relative to a control.

In some embodiments, (a) the locomotor activity and/or energy expenditure is increased by about 5%, 6%, 7%, 8%, 9%, 10%, 11%, 12%, 13%, 14%, 15%, 20%, 25%, 30%, 35%, 40%, 45%, 50%, 60%, 70%, 80%, 90%, or 100%; and/or (b) the subject's body weight and/or food consumption is decreased by 5%, 6%, 7%, 8%, 9%, 10%, 11%, 12%, 13%, 14%, 15%, 20%, 25%, 30%, 35%, 40%, 45%, 50%, 60%, 70%, 80%, 90%, or 100%, relative to a control. In some embodiments, the control is the subject prior to the administering of Pla2g2f.

In a second aspect, the disclosure provides a method of enhancing cognitive function, the method including increasing expression of Pla2g2f in a cell of the subject.

In another aspect, the disclosure provides a method of delaying or reducing cognitive decline in a subject, the method including increasing expression of Pla2g2f in a cell of the subject.

In a further aspect, the disclosure provides a method of improving memory in a subject, the method including increasing expression of Pla2g2f in a cell of the subject.

In another aspect, the disclosure provides a method of treating a neurodegenerative disorder in a subject, the method including increasing expression of Pla2g2f in a cell of the subject or administering Pla2g2f to the subject.

In yet another aspect, the disclosure provides a method of enhancing metabolism in a subject, the method including increasing expression of Pla2g2f in a cell of the subject.

In some embodiments of any of the foregoing aspects, the administration of Pla2g2f or increasing expression of Pla2g2f decreases neuroinflammation in the subject. In some embodiments of any of the foregoing aspects, the cell is a neuron. In some embodiments, the neuron is in the central nervous system of the subject. In some embodiments, the neuron is in the brain of the subject. In some embodiments, the neuron is a dentate granule cell. In some embodiments, the neuron is a neural stem cell. In some embodiments, the cell is a glial cell. In some embodiments, the glial cell is in the central nervous system.

In some embodiments of any one of the foregoing aspects, an expression vector encoding Pla2g2f is administered to the subject. In some embodiments, the expression vector is targeted to the central nervous system. In some embodiments, the expression vector is a viral vector. In some embodiments, the viral vector is an AAV. In some embodiments, the viral vector is a lentiviral vector.

In some embodiments of any one of the foregoing aspects, a nanoparticle including Pla2g2f or a nucleic acid sequence encoding Pla2g2f is administered to the subject. In some embodiments, the nanoparticle is a lipid nanoparticle.

In some embodiments of any one of the foregoing aspects, an exosome comprising Pla2g2f or a sequence encoding Pla2g2f is administered to the subject.

In some embodiments of any of the foregoing aspects, the subject has been diagnosed with memory loss. In some embodiments, the memory loss is due to a neurodegenerative disorder (e.g., Alzheimer's disease (AD) or frontotemporal dementia (FTD)), trauma, or aging.

In some embodiments of any one of the foregoing aspects, the subject has been diagnosed with a neurodegenerative disorder. In some embodiments, the neurodegenerative disorder is Alzheimer's disease (AD), frontotemporal dementia (FTD), Parkinson's disease (PD), or Amyotrophic Lateral Sclerosis (ALS).

In another aspect, the disclosure provides a vector including a nucleic acid sequence encoding Pla2g2f. In a further aspect, the disclosure provides an exosome including Pla2g2f or a nucleic acid sequence encoding Pla2g2f. In an additional aspect, the disclosure provides a nanoparticle, e.g., a lipid nanoparticle, including Pla2g2f or a nucleic acid sequence encoding Pla2g2f. In some embodiments, the nucleic acid sequence encoding Pla2g2f has the sequence of SEQ ID NO: 1 or SEQ ID NO: 2. In some embodiments, the nucleic acid sequence encoding Pla2g2f is at least 70% identical (e.g., at least 75%, 80%, 85%, 90%, 95%, or 99%) to the nucleic acid sequence of SEQ ID NO: 1 or SEQ ID NO: 2. In some embodiments, the nucleic acid sequence encoding Pla2g2f encodes the amino acid sequence of SEQ ID NO: 3 or SEQ ID NO: 4. In some embodiments, the nucleic acid sequence encoding Pla2g2f encodes an amino acid sequence that is at least 70% identical (e.g., at least 75%, 80%, 85%, 90%, 95%, or 99%) to SEQ ID NO: 3 of SEQ ID NO: 4. In some embodiments, the vector is a viral vector. In some embodiments, the viral vector is an AAV or lentiviral vector. In some embodiments of any one of the foregoing aspects, the subject is a human.

In another aspect, the disclosure provides a kit for enhancing cognitive function, improving memory, delaying or reducing cognitive decline, or treating a neurodegenerative disorder in a subject, the kit including a vector including a nucleic acid sequence encoding Pla2g2f and instructions for administration of the vector. In some embodiments, the nucleic acid sequence encoding Pla2g2f has the sequence of SEQ ID NO: 1 or SEQ ID NO: 2. In some embodiments, the nucleic acid sequence encoding Pla2g2f is at least 70% identical (e.g., at least 75%, 80%, 85%, 90%, 95%, or 99%) to the nucleic acid sequence of SEQ ID NO: 1 or SEQ ID NO: 2. In some embodiments, the nucleic acid sequence encoding Pla2g2f encodes the amino acid sequence of SEQ ID NO: 3 or SEQ ID NO: 4. In some embodiments, the nucleic acid sequence encoding Pla2g2f encodes an amino acid sequence that is at least 70% identical (e.g., at least 75%, 80%, 85%, 90%, 95%, or 99%) to SEQ ID NO: 3 of SEQ ID NO: 4. In some embodiments, the vector is a viral vector. In some embodiments, the viral vector is an AAV or lentiviral vector. In some embodiments, the delay or reduction in cognitive decline is in aging.

### BRIEF DESCRIPTION OF THE DRAWINGS

FIG. 1 is a schematic showing the performed steps in a screen for compensatory neuroprotective secreted factors induced by synapse loss. Dentate granule cells (DGCs) were dissected from mDG-Klf9 mice. RNA was isolated from the DGCs and deep sequencing followed by differential gene analysis was performed. One of the most highly upregulated genes was Pla2g2f, which encodes a secreted phospholipase called phospholipase A2 group IIF (Pla2g2f) that is expressed at negligibly low levels only in DGCs of the adult hippocampus and is absent from other regions.

FIG. 2A is a set of photomicrographs showing an increase of Pla2g2f transcripts during aging by in situ hybridization.

FIG. 2B is a graph that quantifies the Pla2g2f transcripts shown in FIG. 2A. Data shown reflect the mean+/−standard deviation (3 animals were used per age group).

FIG. 3A is a schematic depicting the timeline of treatment to delete Pla2g2f in the dentate gyrus of middle-aged (9 months old) Pla2g2f cKO mice.

FIG. 3B is a set of photomicrographs showing immunostaining of a murine dentate gyrus section 5 weeks post retroviral injection of the RV-TdTomato vector.

FIG. 3C is a set of photomicrographs showing immunostaining of dendritic spines of adult-born murine neurons, also at 5 weeks post retroviral injection of the RV-TdTomato vector.

FIG. 3D is a graph that quantifies the number of spines per 20 μm according to FIG. 3B, and data shown reflect the mean+/−standard deviation. Four control mice and five Pla2g2f cKO mice were used.

FIG. 4A is a set of photomicrographs of an immunostaining panel of microglia in different regions within the dentate gyrus of either control of Pla2g2f cKO mice (9 months of age). CD68 was stained as a lysosomal marker, and antibodies for Iba were used to stain microglia. From left to right, molecular layer (ML), granule cell layer (GCL), and Hilus.

FIG. 4B is a set of graphs that quantify the ratio of CD68 punctae to Iba1 punctae (CD68 score). A higher score is associated with aberrant microglia. Data is shown to reflect the mean+/−standard deviation. Seven control mice and five Pla2g2f cKO mice were used.

FIG. 5A is a set of photomicrographs of a GFAP immunostaining of astrocytes in different regions within the dentate gyrus for either control or Pla2g2f cKO mice (9 months of age). From left to right ML, GCL, and Hilus.

FIG. 5B is a set of graphs that quantify the average GFAP punctae per hemisection. Higher GFAP counts are associated with reactive astrocytes. Data is shown to reflect the mean+/−standard deviation. Nine control mice and eight Pla2g2f cKO mice were used.

FIG. 6A is a schematic showing the method of treatment to overexpress Pla2g2f in the dentate gyrus of adult mice using a viral vector.

FIG. 6B is a set of photomicrographs and a graph showing the results of an immunostaining panel for Nestin, an intermediate filament protein that serves as a neural stem cell marker as it is expressed in undifferentiated central nervous system cells during development, and MCM2, a cell proliferation marker, in the dentate gyrus of mice infected with control virus or Pla2g2f overexpression (OE) virus. Viral Pla2g2f overexpression promotes neural stem cell activation and expansion.

FIG. 7A is a schematic showing the method of treatment to overexpress Pla2g2f in the dentate gyrus of adult CX3CR1-GFP mice using a lentiviral vector. Two-month-old mice were injected with CamKIIa-mCherry (Ctrl) or CamKIIa-Pla2g2f-T2a-mCherry (PLA OE) lentiviruses. Five weeks post-injection, microglia population analysis was performed by counting the number of GFP-positive microglia.

FIG. 7B is a set of photomicrographs of a GFP immunostaining assay in Ctrl and PLA OE mice.

FIG. 7C is a set of graphs that quantify the number of GFP-positive cells per hemisection. Data shown reflect the mean+/−standard deviation. Five control mice and four PLA OE mice were used.

FIG. 8A is a schematic showing the object location memory test. 12-month-old CamKIIa-Cre-ERT2 Pla2g2f cKO bigenic mice were intraperitoneally injected with vehicle (Ctrl) or tamoxifen (for Pla2g2f deletion). Mice were then tested for object location memory. Mice were habituated to the empty arena over three days. Mice with Pla2g2f deletion showed impaired spatial memory [discrimination ratio=(time exploring moved object−time exploring unmoved object/time exploring moved object+time exploring unmoved object)]. Data reflect the mean+/−standard error. Ten control mice and seven Pla2g2f cKO mice were used.

FIG. 8B is a graph showing the average distance moved.

FIG. 8C is a graph showing the discrimination ratio, which was calculated as described above. Control data is unshaded and Pla2g2f cKO data is shaded in FIG. 8B and FIG. 8C.

FIG. 9A is a schematic showing the object location memory test. 16-month-old C57BL/6J mice were injected with CamKIIa-mCherry (Ctrl) or CamKIIa-Pla2g2f-T2a-mCherry (PLA OE) lentiviruses and tested for object location memory. Mice were habituated to the empty arena over three days. Overexpression of Pla2g2f in aged mice improves spatial object location memory [discrimination ratio=(time exploring moved objext−time exploring unmoved object/time exploring moved object+time exploring unmoved object)]. Data reflect the mean+/−standard error.

FIG. 9B is a graph showing the average distance moved.

FIG. 9C is a graph showing the discrimination ratio, which was calculated as described above. Control data is unshaded and Pla2g2f cKO data is shaded in FIG. 9B and FIG. 9C.

FIG. 10A is a schematic showing the method of treatment to overexpress Pla2g2f in an Alzheimer's disease (AD) mouse model. 12-13-month-old NL-F/NL-F (referred to as NL-F) AD mice were injected with CamKIIa-mCherry (Ctrl) or CamKIIa-Pla2g2f-T2A-mCherry (PLA OE) lentiviruses. Five weeks later amyloid plaques size was analyzed using 6E10 antibody. Viral overexpression of Pla2g2f in NL-F mice decreases the area of amyloid plaques. Data reflect the mean+/−standard error. Four control mice and four PLA OE mice were used.

FIG. 10B is a set of photomicrographs showing 6E10 immunostaining in Ctrl versus PLA OE mice.

FIG. 10C is a graph that quantifies amyloid plaque size shown in FIG. 10B by measuring the area stained by 6E10. The unshaded bar represents the Ctrl group and the shaded bar represents the PLA OE group.

FIG. 11A is a schematic showing the method of treatment to overexpress Pla2g2f in an Alzheimer's disease (AD) mouse model. 12-14 months old NL-F mice were injected with CamKIIa-mCherry (Ctrl) or CamKIIa-Pla2g2f-T2A-mCherry (PLA OE) lentiviruses. Mice were then tested for Contextual Fear Conditioning. Pla2g2f viral overexpression improves memory retrieval in context A (higher freezing %) 24 h hours after the immediate shock. Data reflect the mean+/−standard error. Four control mice and four PLA OE mice were used.

FIG. 11B upper panel shows the conditions used for Contextual Fear Conditioning. FIG. 11B lower panel is a set of graphs that quantifies memory retrieval and the discrimination ratio (described previously in FIG. 8 and FIG. 9). The lighter shaded bars represent the Ctrl group and the darker shaded bars represent the PLA OE group.

FIG. 12A is a set of photomicrographs showing an immunostaining panel of the dentate gyrus in 12-month-old CamKIIa-Cre-ERT2 Pla2g2f cKO bigenic mice. From left to right: DAPI, Serpina3n, and Serpina3n. Five control mice and four Pla2g2f cKO mice were used.

FIG. 12B is a graph that quantifies the number of Serpina3n punctae in FIG. 12A. Data reflect the mean+/−standard error. The lighter shaded bar represents the control group and the darker shaded bar represents the Pla2g2f cKO group. Loss of Pla2g2f in middle-aged mice results in elevation of Serpina3n.

FIG. 13A is a schematic showing how mice were injected with CamKIIa-mCherry (Ctrl) or CamKIIa-Pla2g2f-T2a-mCherry (PLA OE) lentiviruses in the dentate gyrus before performing a spine analysis. In brief, 16 months old CamKIIa-Cre-ERT2 Pla2g2f cKO bigenic mice were intraperitoneally injected with vehicle (ctrl) or tamoxifen (for Pla2g2f deletion). Two weeks later, both groups were injected in the dentate gyrus with a lentiviral vector expressing mCherry (CamKIIa-mcherry) to label mature dentate granule cells. Four months later, the analysis of dendritic spines of dentate granule cells was performed.

FIG. 13B is a representative image of 20 month old mice injected with vehicle (ctrl) or tamoxifen (for Pla2g2f conditional knockout (PLA cKO)), as described in FIG. 13A.

FIG. 13C is a graphical quantification of the experiment described in FIG. 13A-B. The data shows that the deletion of Pla2g2f reduces dendritic spine density in mature granule cells. Data shown is mean±SEM, n=3 per group. *** p=0.0005.

FIG. 14A is a graph showing the percent body weight of control vs PLA and suggests that overexpression of Pla2g2f reduces body weight in aged mice. In brief, 16 months old C57BL/6 mice were injected with CamKIIa-mCherry (Ctrl) or CamKIIa-Pla2g2f-T2a-mCherry (PLA overexpression) lentiviruses in the dentate gyrus, and each mouse's body weight was measured before and two weeks after the injection. % body weight loss is [(body weight after injection−body weight before injection)/body weight before injection]×100. Data shown is the mean±SEM, n=9 per group. ** p=0.0094 (% body weight loss Ctrl vs PlaOE); ***p=0.0008 (body weight in grams).

FIG. 14B is a graph showing the percent body weight of control vs control (pre- and post-injection). In brief, 16 months old C57BL/6 mice were injected with CamKIIa-mCherry (Ctrl) lentiviruses in the dentate gyrus, and each mouse's body weight was measured before and two weeks after the injection. % body weight loss is [(body weight after injection−body weight before injection)/body weight before injection]×100. Data shown is the mean±SEM, n=9 per group. ** p=0.0094 (% body weight loss Ctrl vs PlaOE); ***p=0.0008 (body weight in grams).

FIG. 14C is a graph showing the percent body weight of PLA vs PLA. In brief, 16 months old C57BL/6 mice were injected with CamKIIa-Pla2g2f-T2a-mCherry (PLA overexpression) lentiviruses in the dentate gyrus, and each mouse's body weight was measured before and two weeks after the injection. % body weight loss is [(body weight after injection−body weight before injection)/body weight before injection]×100. Data shown is the mean±SEM, n=9 per group. ** p=0.0094 (% body weight loss Ctrl vs PlaOE); ***p=0.0008 (body weight in grams before and after lentiviral injection PIa vs PIa).

FIG. 15 is a graph showing the effects of Pla2g2f over expression on metabolism. The graph represents locomotor activity over time in control and PLA overexpressed mice. In brief, three weeks after the lentiviral injection (described in FIG. 13A), mice were placed into metabolic cages (Promethion System) for five days to measure basal locomotor activity over time. Food and water were provided ad libitum. Pla2g2f overexpression resulted in increased locomotor activity at night but not during the day.

FIG. 16 is a graph showing the effects of Pla2g2f over expression on metabolism. The graph represents total body mass over time in control and PLA overexpressed mice. In brief, three weeks after the lentiviral injection (described in FIG. 13A), mice were placed into metabolic cages (Promethion System) for five days to measure body mass over time. Food and water were provided ad libitum. Pla2g2f overexpression resulted in reduced body weight.

FIG. 17 is a graph showing the effects of Pla2g2f over expression on metabolism. The graph represents total food consumption over time in control and PLA overexpressed mice. In brief, three weeks after the lentiviral injection (described in FIG. 13A), mice were placed into metabolic cages (Promethion System) for five days to measure food intake over time. Food and water were provided ad libitum. Pla2g2f overexpression resulted in reduced food consumption.

FIG. 18 is a graph showing the effects of Pla2g2f over expression on metabolism. The graph represents total energy expenditure over time in control and PLA overexpressed mice. In brief, three weeks after the lentiviral injection (described in FIG. 13A), mice were placed into metabolic cages (Promethion System) for five days to measure energy expenditure over time. Food and water were provided ad libitum. Pla2g2f overexpression resulted in enhanced energy expenditure.

FIG. 19 shows a schematic (left) illustrating the experimental design of human forebrain organoids infected with CamKIIa-mCherry (Ctrl) or CamKIIa-Pla2g2f-T2a-mCherry (PLA OE) lentiviruses at DIV (Day in Vitro) 60. A representative image (middle) of human microglia following lentivirus infections shows that human microglia differentiated from human induced pluripotent stem cells. Microglia were then treated with oleic acid (an inflammatory stimulus) for 24 hours to induce lipid droplets formation. Lipid droplets were visualized and quantified (right) using BODIPY staining. Conditioned medium derived from human forebrain organoids Ctrl or overexpressing Pla2g2f was applied to lipid-burdened human microglia. *p value<0.05; n=6 Ctrl, n=9 Pla2g2f overexpression. Pla2g2f expression in neuronal organoids reduced inflammation associated lipid droplets in microglia.

FIG. 20A is a principal component analysis (PCA) of the lipidome in Ctrl and Pla2g2f cKO mice. In brief, 16 month old Pla2g2f cKO and C57BL/6 mice were injected with CamKIIa Cre-GFP lentivirus. The dentate gyrus was dissected 5 weeks after the lentiviral injection, and the tissue was homogenized in phosphate-buffered saline using Bead Mill Homogenizer (VWR). Subsequently, lipids were extracted according to Folch's Method. The organic phase of each sample, normalized by tissue weight, was then separated using ultra-high performance liquid chromatography coupled to tandem mass spectrometry (UHPLC-MSMS) method. Shown circled are the Z scores clusters of a lipid-species distribution of all lipid species identified in the lipidome of Ctrl and Pla2g2f cKO mice. These data support that loss of Pla2g2f in aging mice disrupts lipid homeostasis.

FIG. 20B is a distribution plot of lipid species in Ctrl and Pla2g2f cKO mice. In brief, 16 month old Pla2g2f cKO and C57BL/6 mice were injected with CamKIIa Cre-GFP lentivirus. The dentate gyrus was dissected 5 weeks after the lentiviral injection, and the tissue was homogenized in phosphate-buffered saline using Bead Mill Homogenizer (VWR). Subsequently, lipids were extracted according to Folch's Method. The organic phase of each sample, normalized by tissue weight, was then separated using ultra-high performance liquid chromatography coupled to tandem mass spectrometry (UHPLC-MSMS) method. The fractional abundance of lipid species is relative to total lipid amount.

FIG. 20C is bar graph of the lipidome of Ctrl and Pla2g2f cKO mice averaged by condition. In brief, 16 month old Pla2g2f cKO and C57BL/6 mice were injected with CamKIIa Cre-GFP lentivirus. The dentate gyrus was dissected 5 weeks after the lentiviral injection, and the tissue was homogenized in phosphate-buffered saline using Bead Mill Homogenizer (VWR). Subsequently, lipids were extracted according to Folch's Method. The organic phase of each sample, normalized by tissue weight, was then separated using ultra-high performance liquid chromatography coupled to tandem mass spectrometry (UHPLC-MSMS) method. Lipid molecules were considered differentially expressed if they met a p-value cut-off <0.05 and the log 2 fold change cut-off 1.15. n=5 Ctrl, n=3 Pla2g2f cKO.

FIG. 21A is heatmap of the abundance and composition of lipid species in Ctrl and Pla2g2f cKO mice. In brief, 16 month old Pla2g2f cKO and C57BL/6 mice were injected with CamKIIa Cre-GFP lentivirus. The dentate gyrus was dissected 5 weeks after the lentiviral injection, and the tissue was homogenized in phosphate-buffered saline using Bead Mill Homogenizer (VWR). Subsequently, lipids were extracted according to Folch's Method. The organic phase of each sample, normalized by tissue weight, was then separated using ultra-high performance liquid chromatography coupled to tandem mass spectrometry (UHPLC-MSMS) method.

FIG. 21B is graph representing fractional abundance of lipid species in Ctrl and Pla2g2f cKO mice. In brief, 16 month old Pla2g2f cKO and C57BL/6 mice were injected with CamKIIa Cre-GFP lentivirus. The dentate gyrus was dissected 5 weeks after the lentiviral injection, and the tissue was homogenized in phosphate-buffered saline using Bead Mill Homogenizer (VWR). Subsequently, lipids were extracted according to Folch's Method. The organic phase of each sample, normalized by tissue weight, was then separated using ultra-high performance liquid chromatography coupled to tandem mass spectrometry (UHPLC-MSMS) method.

### DEFINITIONS

As used herein, the term “about” refers to a value that is within 10% above or below the value being described.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, and shrimp AAV. See, e.g., Fields et al. Virology, 4^{th} ed. Lippincott-Raven Publishers, Philadelphia, 1996. Additional AAV serotypes and classes have been identified recently. (See, e.g., Gao et al. J. Virol. 78:6381 (2004); Moris et al. Virol. 33:375 (2004). The genomic sequences of various serotypes of AAV, as well as the sequences of the native ITRs, Rep proteins, and capsid subunits are known in the art. Such sequences may be found in the literature or in public databases such as GenBank. See, e.g., GenBank Accession Numbers NC-002077, NC-001401, NC-001729, NC-001863, NC-001829, NC-001862, NC-000883, NC-001701, NC-001510, NC-006152, NC-006261, AF063497, U89790, AF043303, AF028705, AF028704, J02275, J01901, J02275, X01457, AF288061, AH009962, AY028226, AY028223, AY631966, AX753250, EU285562, NC-001358, NC-001540, AF513851, AF513852 and AY530579; the disclosures of which are incorporated by reference herein for teaching AAV nucleic acid and amino acid sequences. See also, e.g., Bantel-Schaal et al. J. Virol. 73:939 (1999); Chiorini et al. J. Virol. 71:6823 (1997); Chiorini et al. J. Virol. 73:1309 (1999); Gao et al. Proc. Nat. Acad. Sci. USA 99:11854 (2002); Moris et al. Virol. 33:375 (2004); Muramatsu et al. Virol. 221:208 (1996); Ruffing et al. J. Gen. Virol. 75:3385 (1994); Rutledge et al. J. Virol. 72:309 (1998); Schmidt et al. J. Virol. 82:8911 (2008); Shade et al. J. Virol. 58:921 (1986); Srivastava et al. J. Virol. 45:555 (1983); Xiao et al. J. Virol. 73:3994 (1999); WO 00/28061, WO 99/61601, WO 98/11244; and U.S. Pat. No. 6,156,303; the disclosures of which are incorporated by reference herein for teaching AAV nucleic acid and amino acid sequences.

As used herein, the term “aging” refers to progressive physiological changes in an organism that lead to senescence, or a decline or biological functions and of the organism's ability to adapt to metabolic stress. Aging occurs as the accumulation of diverse deleterious changes that naturally occur in cells and tissues with advancing age pose an increased risk of disease and death. By “delaying cognitive decline in aging,” it is meant that one or more effects of aging on cognitive function, such as a decline in cognitive function or a loss of memory, are suppressed or ameliorated. A decline or improvement in cognitive function can be measured by a change, or lack of change, in performance on a cognitive test. Non-limiting examples of cognitive tests that can be used to assess cognitive function include AD8, AWV, GPCOG, HRA, MIS, MMSE, MoCA, SLUMS, and Short IQCODE.

As used herein, the terms “amyotrophic lateral sclerosis” and “ALS”, also called Lou Gehrig's disease, refer to a fatal disease affecting motor neurons of the cortex, brain stem and spinal cord. In the context of the present invention the term “ALS” includes the spectrum of neurodegenerative disorders known under the names of Classical (Charcot's) ALS, Lou Gehrig's disease, motor neuron disease (MND), progressive bulbar palsy (PBP), progressive muscular atrophy (PMA), primary lateral sclerosis (PLS), bulbar onset ALS, spinal onset ALS and ALS with multi-system involvement (Wijesekera L C and Leigh P N. Amyotrophic lateral sclerosis. Orphanet J. Rare Dis. 2009, 4:3).

As used herein, “Alzheimer's disease” and “AD” refer to a late-onset neurodegenerative disorder presenting as cognitive decline, loss of short- and long-term memory, attention deficits, language-specific problems, disorientation, impulse control, social withdrawal, anhedonia, and other symptoms. Brain tissue of AD patients exhibits neuropathological features such as extracellular aggregates of amyloid-3 protein and neurofibrillary tangles of hyperphosphorylated microtubule-associated tau proteins. Accumulation of these aggregates is associated with neuronal loss and atrophy in a number of brain regions including the frontal, temporal, and parietal lobes of the cerebral cortex as well as subcortical structures like the basal forebrain cholinergic system and the locus coeruleus within the brainstem. AD is also associated with increased neuroinflammation characterized by reactive gliosis and elevated levels of pro-inflammatory cytokines.

The term “codon” as used herein refers to any group of three consecutive nucleotide bases in a given messenger RNA molecule, or coding strand of DNA, that specifies a particular amino acid or a starting or stopping signal for translation. The term codon also refers to base triplets in a DNA strand.

As used herein, the term “cognitive function” refers collectively to a variety of mental processes and abilities such as perception, attention, memory, decision making, and language comprehension. A decline or improvement in cognitive function can be measured by a change, or lack of change, in performance on a cognitive test. Non-limiting examples of cognitive tests that can be used to assess cognitive function include AD8, AWV, GPCOG, HRA, MIS, MMSE, MoCA, SLUMS, and Short IQCODE. Cognitive function can also be qualitatively assessed by a subject or patient's self-perceived observation of memory loss.

Throughout this specification and claims, the word “comprise,” or variations such as “comprises” or “comprising,” will be understood to imply the inclusion of a stated integer or group of integers but not the exclusion of any other integer or group of integers.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in Table 1, below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium at | |
| | | | | pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large,
and >200 is bulky

From this table it is appreciated that the conservative amino acid families include, e.g., (i) G, A, V, L, I, P and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “effective amount,” “therapeutically effective amount,” and a “sufficient amount” of a therapeutic described herein (e.g., Pla2g2f) refer to a quantity sufficient to, when administered to the subject, including a mammal, for example a human, effect beneficial or desired results, including clinical results. As such, an “effective amount” or synonym thereof depends upon the context in which it is being applied. For example, in the context of treating neurodegeneration, it is an amount of a therapeutic sufficient to achieve a treatment response as compared to the response obtained without administration of the therapeutic. The amount of a given therapeutic described herein (e.g., Pla2g2f) that will correspond to such an amount will vary depending upon various factors, such as the pharmaceutical formulation, the identity of the subject (e.g., age, sex, weight), and the like, but can nevertheless be routinely determined by one skilled in the art. Also, as used herein, a “therapeutically effective amount” of a therapeutic of the present disclosure (e.g., Pla2g2f) is an amount which results in a beneficial or desired result in a subject as compared to a control.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “enhancing the metabolism” or “enhanced metabolism” is used to refer to an increase in catabolism and/or anabolism in a subject that results in an increase in a subject's locomotor activity (e.g., as measured by radio-frequency identification (RFID) technology and the like), a decrease in a subject's body weight, a decrease in a subject's food consumption (e.g., as measured by calorie intake), and/or an increase in the subject's energy expenditure (e.g., as measured in calories, e.g., kcal), relative to a control, e.g., the subject prior to the administering of Pla2g2f. Any increase in locomotor activity, decrease in body weight, decrease in food consumption, or increase in energy expenditure (e.g., about a 5%, 6%, 7%, 8%, 9%, 10%, 11%, 12%, 13%, 14%, 15%, 20%, 25%, 30%, 35%, 40%, 45%, 50%, 60%, 70%, 80%, 90%, 100%, increase or more) relative to a control (e.g., a subject that was not administered Pla2g2f or a subject prior to administering Pla2g2f) constitutes an enhanced metabolism.

As used herein, the term “exosome” refers to extracellular vesicles that are released from cells upon fusion of an intermediate endocytic compartment, the multivesicular body, with the plasma membrane. Exosomes play a role in cell-to-cell communication. Their size and ability to distribute through vastly complex body fluids makes them desirable carriers for therapeutics.

As used herein, the term “expression vector” refers to a vector, such as a plasmid, yeast, or animal virus genome, used to introduce foreign genetic material into a host cell in order to replicate and amplify the foreign DNA sequences as a recombinant molecule.

As used herein, the terms “frontotemporal dementia” and “FTD” refer to a disorder caused by the degeneration of the brain's frontal lobes and degeneration that may extend to the temporal lobe. FTD is one of the three syndromes caused by frontotemporal lobe degeneration and the second most common cause of early dementia, after AD. Diagnostic criteria according to the Lund-Manchester criteria include onset and gradual progression, early decline in social interpersonal conduct, early impairment in regulation of personal conduct, early emotional blunting, and early loss of insight. Symptoms of FTD may appear between the ages of about 45 to about 65 years (e.g., 50 to about 60) of age. As used herein, “FTD” is intended to include all the stages (such as preclinical stage) and subtypes of the disease.

As used herein, the term “GC content” refers to the quantity of nucleosides in a particular nucleic acid molecule, such as a DNA or RNA polynucleotide, that are either guanosine (G) or cytidine (C) relative to the total quantity of nucleosides present in the nucleic acid molecule. GC content may be expressed as a percentage, for instance, according to the following formula:

GC Content=((Total quantity of guanosine nucleosides)+(Total quantity of cytidine nucleosides)/(Total quantity of nucleosides))×100

As used herein, the term “memory” refers to the faculty by which the mind stores and remembers information. Memory can be assessed by cognitive function tests that specifically appraise “working memory,” or the part of short-term memory that is concerned with immediate conscious perceptual and linguistic processing. Working memory is held in the mind for short periods of time and used in the execution of cognitive tasks, e.g., keeping an address in mind while being given directions. A decline or improvement in memory can be measured by a change, or lack of change, in performance on a memory test. For example, according to the methods of the disclosure, “improving memory” refers to an increase in performance on a memory test. Non-limiting examples of memory tests that can be used to assess working memory include AD8, AWV, GPCOG, MIS, Mini-cog, MMSE, MoCA, SLUMS, and Short IQCODE. The increase in performance on the memory test may be a 10% increase, a 15% increase, a 25% increase, or a 50% increase in the score.

As used herein, the term “modified nucleotide” refers to a nucleotide or portion thereof (e.g., adenosine, guanosine, thymidine, cytidine, or uridine) that has been altered by one or more enzymatic or synthetic chemical transformations. Exemplary alterations observed in modified nucleotides described herein or known in the art include the introduction of chemical substituents, such as halo, thio, amino, azido, alkyl, acyl, or other functional groups at one or more positions (e.g., the 2′, 3′, and/or 5′ position) of a 2-deoxyribonucleotide or a ribonucleotide.

As used herein, the term “mutation” refers to a change in the nucleotide sequence of a gene (e.g., Pla2g2f) or a change in the polypeptide sequence of a protein (e.g., Pla2g2f). Mutations in a gene or protein may occur naturally as a result of, for example, errors in DNA replication, DNA repair, irradiation, and exposure to carcinogens or mutations may be induced as a result of administration of a transgene expressing a mutant gene. Mutations may result from single or multiple nucleotide insertions, deletions, or substitutions.

“Neurodegeneration” refers to the progressive loss of the number, structure, and/or function of neurons. In some instances, a patient having neurodegeneration may be diagnosed by assessing their performance on a cognitive test or by another qualitative assessment of general risk factors, such as aging. Exemplary, non-limiting symptoms of neurodegeneration include declining cognitive function, such as a deficit in, e.g., complex attention, executive function, learning and memory, language, perceptual-motor function, and social cognition. Cognitive tests can be used to assess cognitive function in patients displaying one or more symptoms or neurodegeneration.

As used herein, the terms “neurodegenerative disorder” and “neurodegenerative disease” refer interchangeably to a disorder characterized by progressive loss of the number (e.g., by cell death), structure, and/or function of neurons. In some instances, a neurodegenerative disease may be associated with genetic defects, protein misfolding, defects in protein degradation, programmed cell death, membrane damage, or other processes. Exemplary, non-limiting neurodegenerative disorders include AD and ALS.

As used herein, the term “neuroinflammation” refers to a process characterized by reactive gliosis and elevated levels of pro-inflammatory cytokines in the nervous system. Neuroinflammation results in a sustained increase of inflammatory microglia and reactive astrocytes in the nervous system and contributes to the progression of aging and neurodegenerative disorders.

As used herein, “neuron” refers to a type of cell found in the brain and nervous system. Neurons are responsible for receiving sensory input from the external world, for sending motor commands to muscles, and for transforming and relaying electrical signals between neurons connected by neural networks. These cells contain three distinct parts, including a cell body, an axon, and dendrites.

“Nucleic acid” or “polynucleotide,” as used interchangeably herein, refer to polymers of nucleotides of any length and include DNA and RNA.

As used herein, the term “nanoparticle” refers to a particle which is between 1-100 nanometers (nm) in size. Nanoparticles can be used to encapsulate a therapeutic agent and deliver it to target cells, and as such are used as drug delivery systems. Non-limiting examples of nanoparticles include lipid nanoparticles, metallic nanoparticles, and silica-based nanoparticles.

As used herein, the terms “Parkinson's disease” and “PD” refer to a neurodegenerative disease characterized by motor and non-motor symptoms. Exercise symptoms mainly include dyskinesias, hypotonia, stiffness and progression, where exercise dyskinesia includes exercise-induced relaxation and even anemia. Non-motor symptoms include pain, constipation, delayed gastric emptying, depression, and sleep disorders.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The terms “polyadenylation signal,” “polyadenylation site,” and “pA” are used interchangeably to herein to mean a nucleic acid sequence sufficient to direct the addition of polyadenosine ribonucleic acid to an RNA molecule expressed in a cell.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “phospholipase A group IIF” and “Pla2g2f” refer to a phospholipase whose non-neuronal roles acting on distinct classes of membrane phospholipids to govern stem cell homeostasis, inflammation, and macrophage polarization. Pla2g2f can be expressed in the brain. The terms “phospholipase A group IIF” and “Pla2g2f” also refer to variants of wild-type Pla2g2f peptides and nucleic acids encoding the same, such as variant proteins having at least 70% sequence identity (e.g., at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to the amino acid sequence of a wild-type Pla2g2f peptide (e.g., SEQ ID NO: 3 or SEQ ID NO: 4) or polynucleotides having at least 70% sequence identity (e.g., at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to the nucleic acid sequence of a wild-type Pla2g2f gene (e.g., SEQ ID NO: 1 or SEQ ID NO: 2), provided that the Pla2g2f analog encoded retains the therapeutic function of wild-type Pla2g2f. Pla2g2f is conserved in both function and amino acid sequence (with at least 70% amino acid sequence identity to the wildtype Pla2g2f amino acid sequence of SEQ ID NO: 4) across species. The Pla2g2f nucleotide sequence is not as highly conserved across species.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nat. Rev. Genet. 8:424 (2007), the disclosure of which is incorporated herein by reference as it pertains to nucleic acid regulatory elements.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “tissue” refers to a group of cells that have a similar structure and/or act together to perform a specific function.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the term “transgene” refers to a recombinant nucleic acid (e.g., DNA or cDNA) encoding a gene product (e.g., Pla2g2f). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s) and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “subject” and “patient” refer to an animal (e.g., a mammal, such as a human). A subject to be treated according to the methods described herein may be one having neuroinflammation and/or neurodegeneration, such as one who has been diagnosed a neurodegenerative disorder (e.g., AD, FTD, PD, or ALS) or one who is displaying one or more symptoms of neurodegeneration, e.g., declining cognitive function. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition, such as aging.

As used herein, the terms “transduction” and “transduce” refer to a method of introducing a viral vector construct or a part thereof into a cell, and subsequent expression of a transgene, such as a Pla2g2f transgene, encoded by the vector construct or part thereof in the cell.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, abbreviations of gene names refer to a wild-type version of the corresponding gene, as well as variants (e.g., splice variants, truncations, concatemers, and fusion constructs, among other) thereof. Examples of such variants are genes having at least 70% sequence identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity) to any of the nucleic acid sequences of a wild-type version of the gene.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of Pla2g2f as described herein include plasmids that contain regulatory sequences, such as promoter and enhancer regions, which direct gene transcription. Other useful vectors for expression of Pla2g2f contain polynucleotide sequences that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

### DETAILED DESCRIPTION

Hippocampal dysfunction is a hallmark of age-related cognitive decline and Alzheimer's disease (AD). Studies in aged rodents, non-human primates and humans (aged or with Mild Cognitive Impairment, MCI) document synapse loss, reduced inhibitory neuron structural plasticity, increased neuronal excitability and reduced neurogenesis in the hippocampal entorhinal cortex (EC)-dentate gyrus (DG)-CA3 circuit (Haberman, R. P., Branch, A., and Gallagher, M. (2017) Neurotherapeutics 14, 662-676).

But aging and AD does not only affect neurons. Instead, aging also polarizes microglia and astrocytes into reactive and inflammatory states. Specifically, inflammatory and interferon responsive microglia are profoundly increased during aging and AD in the brain (Clarke, et al., (2018) Proc Natl Acad Sci USA 115, E1896-E1905). Specifically, inflammatory and interferon responsive microglia (expressing Lgals3, cystatin F, chemokines Ccl4 and Ccl3, and inflammatory cytokine interleukin 1 Beta) (Hammond, et al. (2019) Immunity 50, 253-271 e256) are profoundly increased during aging in the brain.

Additionally, lipid droplet associated microglia (expressing Ccl3, Cxcl10, 11-6) that exhibit impaired phagocytosis are dramatically increased in the aged DG (Hammond, et al. (2019) Immunity 50, 253-271 e256; Marschallinger, et al. (2020) Nat Neurosci). Proinflammatory microglia, in turn, are thought to induce A1 astrocytes by secreting cytokines and complement factors (Liddelow, et al. (2017) Nature 541, 481-487). A1 astrocytes, unlike astrocytes in adulthood, are neurotoxic, exhibit impaired phagocytosis, impede synapse formation and function (Liddelow, et al. (2017) Nature 541, 481-487).

Described herein are compositions and methods for the treatment of neuroinflammation and neurodegeneration (e.g., a neurodegenerative disease), such as Alzheimer's disease (AD), frontotemporal dementia (FTD), Parkinson's disease (PD), and Amyotrophic Lateral Sclerosis (ALS), or one or more (e.g., two, three, four, or five) symptoms of neurodegeneration, including declining cognitive function and memory loss, in a subject (e.g., a human) in need thereof. The compositions and methods described herein are useful for increasing expression of the human phospholipase A2 group IIF (Pla2g2f) protein and for treating neurodegeneration and neuroinflammation. Exemplary compositions described herein include an adeno-associated virus (AAV) encoding Pla2g2f for expression of Pla2g2f protein in a cell (e.g., a neuron). Without being limited by mechanism, the compositions described herein may ameliorate pathology associated with neuroinflammation and neurodegeneration (e.g., a neurodegenerative disorder, e.g., AD, FTD, PD, or ALS, or one or more symptoms of neurodegeneration, e.g., declining cognitive function) by efficaciously stimulating the expression of the human Pla2g2f protein. Using the compositions and methods described herein, one can treat neuroinflammation and neurodegeneration in a subject (e.g., a human subject) by administering an expression vector (e.g., an AAV vector or a lentiviral vector), a nanoparticle (e.g., a lipid nanoparticle), or exosome described herein.

The present invention is based, at least in part, on the discovery that increasing Pla2g2f expression can reduce and/or ameliorate symptoms of neuroinflammation and neurodegeneration, including, but not limited to, declining cognitive function and memory loss.

### Methods of Treating Neurodegeneration

Using the compositions and methods described herein, a patient having memory loss, neuroinflammation and/or neurodegeneration, such as a patient diagnosed with a neurodegenerative disorder or displaying one or more symptoms of neurodegeneration, e.g., cognitive decline due to aging, can be administered a composition that increases expression of Pla2g2f. In one embodiment, a nucleic acid molecule that includes a transgene encoding Pla2g2f, or a composition encoding the same, so as to increase expression of Pla2g2f, is administered. Without being limited by mechanism, this increase in expression provides the beneficial effect of boosting Pla2g2f-mediated hydrolysis of extracellular surface phospholipids while also enhancing Pla2g2f-mediated reduction of microglia density, reactive astrocyte density, and amyloid burden, and increasing Pla2g2f-mediated synapse maintenance. Subsequently, symptoms of neuroinflammation and neurodegeneration are ameliorated.

In some embodiments, the patient has been diagnosed with or is displaying one or more symptoms of a neurodegenerative disorder. In some embodiments, the neurodegenerative disorder is AD. In some embodiments, the neurodegenerative disorder is ALS. In some embodiments, the neurodegenerative disorder is FTD. In some embodiments, the neurodegenerative disorder is PD.

In some embodiments, the patient has neurodegeneration or is displaying one or more symptoms of neurodegeneration. For example, in some embodiments, the one or more symptoms of neurodegeneration may include, but are not limited to, declining cognitive function, loss of memory, neuroinflammation (e.g., an increase in microglia, an increase in reactive astrocytes, or an increase in amyloid plaques or in amyloid plaque size), and synapse loss. In some embodiments, the neurodegeneration is characterized by a combination of any two or more symptoms described herein.

### Neurodegenerative Disorders

Neurodegenerative disorders are defined as a collection of disorders that feature cognitive impairment as a core symptom and that show cognitive decline relative to a previously higher level of cognition (e.g., acquired impairment), rather than a developmental impairment. Neurodegenerative disorders can be categorized on the basis of their etiological origin. For example, non-limiting examples of neurodegenerative disorders may include neurodegenerative disorders due to AD, neurodegenerative disorders due to FTD, neurodegenerative disorders due to PD, neurodegenerative disorders due to ALS, neurodegenerative disorders due to aging, neurodegenerative disorders due to another medical condition, neurodegenerative disorders due to multiple etiologies, and unspecified neurodegenerative disorders.

The compositions and methods disclosed herein are useful for the treatment of neuroinflammation and neurodegenerative disorders and/or symptoms of neurodegeneration in patients (e.g., human patients) in need thereof.

### Improvement in Cognitive Function

The beneficial treatment effects of the compositions and methods described herein, such as the ability of the methods and compositions described herein to express or increase expression of Pla2g2f and subsequently improve one or more symptoms of neurodegeneration may manifest clinically in a variety of ways. For example, patients having neurodegeneration, such as a patient diagnosed with or displaying one or more symptoms of neurodegeneration, e.g., cognitive decline, may exhibit an improvement in cognitive function such as an improvement in memory. The improvement in cognitive function may be observed, for example, as an improvement in performance on a test to assess cognitive function, such as, but not limited to, AD8, AWV, GPCOG, HRA, MIS, MMSE, MoCA, SLUMS, and Short IQCODE, which are all known in the art. For example, using the compositions and methods described herein, a patient having neurodegeneration, such as a patient diagnosed with or displaying one or more symptoms of neurodegeneration, e.g., cognitive decline, may exhibit an improvement in memory as seen by an improvement in performance on a MoCA test, such as by retaining a greater number of items than in a previous performance conducted, for example, prior to or at the start of treatment.

### Reduction in Neuroinflammation

Additionally, in patients having neurodegeneration, such as a patient diagnosed with or displaying one or more symptoms of neurodegeneration, e.g., neuroinflammation, the beneficial therapeutic effects of the methods and compositions described herein may manifest as a reduction in neuroinflammation (e.g., a reduction in the number of microglia and/or reactive astrocytes, a reduction in the rate of synapse loss, or a reduction in amyloid burden, e.g., a reduction in the number and/or size of amyloid plaques). Evaluation of neuroinflammation may be performed in any number of methods known in the art, such as, but not limited to, by immunostaining for microglial, reactive astrocyte, synapse, and amyloid plaque markers on a biopsy. Thus, using the compositions and methods described herein, a patient having neurodegeneration, such as a patient diagnosed with or displaying one or more symptoms of neurodegeneration, e.g., neuroinflammation, may be administered a Pla2g2f vector or composition encoding the same so as to reduce and/or ameliorate neuroinflammation.

### Methods of Treatment

Subjects that may be treated as described herein are subjects having neurodegeneration (e.g., a neurodegenerative disorder such as AD, FTD, PD, or ALS) or displaying one or more symptoms of neurodegeneration, e.g., declining cognitive function, loss of memory, or neuroinflammation. The compositions and methods described herein may also be used as a preventative treatment to patients at risk of developing neurodegeneration (e.g., a neurodegenerative disorder such as AD, FTD, PD, or ALS, or one or more symptoms of neurodegeneration, e.g., declining cognitive function or neuroinflammation), such as, but not limited to, aging patients (e.g., patients above the age of 70). Patients at risk for neurodegeneration may show early symptoms of neurodegeneration (e.g., memory loss). Furthermore, the methods described herein may be used to enhance the metabolism (e.g., locomotor activity, body weight or mass, food consumption, energy expenditure) and/or reduce body weight of a subject, such as a subject described herein.

In some embodiments, the disclosure provides a method of treating neurodegeneration (e.g., a neurodegenerative disorder such as AD, FTD, PD, or ALS, or one or more symptoms of neurodegeneration, e.g., declining cognitive function or neuroinflammation) in a human patient in need thereof. In some embodiments, the patient is an aging patient (e.g., a human patient above the age of 70).

In some embodiments, the disclosure provides a method of improving cognitive function in a human patient diagnosed as having neurodegeneration (e.g., a neurodegenerative disorder such as AD, FTD, PD, or ALS, or one or more symptoms of neurodegeneration, e.g., declining cognitive function). In some embodiments, the patient is a human patient above the age of 70.

In some embodiments, the disclosure provides a method of restoring Pla2g2f expression in the frontal cortex of a human patient diagnosed as having neurodegeneration (e.g., a neurodegenerative disorder such as AD, FTD, PD, or ALS, or one or more symptoms of neurodegeneration, e.g., declining cognitive function). In some embodiments, the patient is a human patient above the age of 70.

In some embodiments, the disclosure provides a method of enhancing the metabolism of a subject, the method comprising administering Pla2g2f to the subject or increasing expression of Pla2g2f in a cell of the subject. In some embodiments, enhancing the metabolism is exhibited by: (a) increased locomotor activity; (b) decreased body weight; (c) decreased food consumption; and/or (d) increased energy expenditure, relative to a control. In some embodiments, (a) the locomotor activity and/or energy expenditure is increased by about 5%, 6%, 7%, 8%, 9%, 10%, 11%, 12%, 13%, 14%, 15%, 20%, 25%, 30%, 35%, 40%, 45%, 50%, 60%, 70%, 80%, 90%, or 100%; and/or (b) the subject's body weight and/or food consumption is decreased by 5%, 6%, 7%, 8%, 9%, 10%, 11%, 12%, 13%, 14%, 15%, 20%, 25%, 30%, 35%, 40%, 45%, 50%, 60%, 70%, 80%, 90%, or 100%, relative to a control. In some embodiments, the control is the subject prior to the administering of Pla2g2f.

### Delivery

### I. Viral Vectors for Expression of Therapeutic Nucleic Acid Molecules

Viral genomes provide a rich source of vectors that can be used for the efficient delivery of exogenous genes into a mammalian cell (e.g., a neuron). Viral genomes are particularly useful vectors for gene delivery as the nucleic acids contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox).

Other viruses include Norwalk virus, togavirus, flavivirus, reoviruses, papovavirus, hepadnavirus, human papilloma virus, human foamy virus, and hepatitis virus, for example. Examples of retroviruses are avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996)). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### IA. Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, J. Gene Med. 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral complimentary DNA (cDNA) deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally may include a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below. The Lentigen LV described in Lu et al., J. Gene Med. 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther. 8:811 (2001), Osborn et al., Mol. Ther. 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may be a clinical grade vector.

### IB. Adeno-Associated Viral Vectors

Nucleic acids of the compositions and methods described herein may be incorporated into a recombinant linear adeno-associated virus (rAAV) vector, a recombinant self-complementary AAV (scAAV) vector, and/or virions, in order to facilitate their introduction into a cell (e.g., a neuron). Adeno-associated virus (AAV) vectors can be used in the central nervous system, and appropriate promoters and serotypes are discussed in Pignataro et al., J Neural Transm., 125: 575 (2018), the disclosure of which is incorporated herein by reference as it pertains to promoters and AAV serotypes useful in CNS gene therapy. In some embodiments, the AAV is a single-stranded rAAV. In some embodiments, the AAV is a scAAV.

rAAV vectors useful in the compositions and methods described herein are recombinant nucleic acid constructs (e.g., nucleic acids capable of expression in muscle cells or neurons) that include (1) a heterologous sequence to be expressed and (2) viral sequences that facilitate integration and expression of the heterologous genes. The viral sequences may include those sequences of AAV that are required in cis for replication and packaging (e.g., functional inverted terminal repeat sequences (ITR)) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. In some embodiments, the AAV ITR is an AAV2 ITR. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

In some embodiments, the AAV includes a nucleic acid sequence that is at least 70% (e.g., at least 75%, 80%, 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 1. For example, in some embodiments, the AAV includes a nucleic acid sequence that is at least 75% identical to the nucleic acid sequence of SEQ ID NO: 1. In some embodiments, the AAV includes a nucleic acid sequence that is at least 80% identical to the nucleic acid sequence of SEQ ID NO: 1. In some embodiments, the AAV includes a nucleic acid sequence that is at least 85% identical to the nucleic acid sequence of SEQ ID NO: 1. In some embodiments, the AAV includes a nucleic acid sequence that is at least 86% identical to the nucleic acid sequence of SEQ ID NO: 1. In some embodiments, the AAV includes a nucleic acid sequence that is at least 87% identical to the nucleic acid sequence of SEQ ID NO: 1. In some embodiments, the AAV includes a nucleic acid sequence that is at least 88% identical to the nucleic acid sequence of SEQ ID NO: 1. In some embodiments, the AAV includes a nucleic acid sequence that is at least 89% identical to the nucleic acid sequence of SEQ ID NO: 1. In some embodiments, the AAV includes a nucleic acid sequence that is at least 90% identical to the nucleic acid sequence of SEQ ID NO: 1. In some embodiments, the AAV includes a nucleic acid sequence that is at least 91% identical to the nucleic acid sequence of SEQ ID NO: 1. In some embodiments, the AAV includes a nucleic acid sequence that is at least 92% identical to the nucleic acid sequence of SEQ ID NO: 1. In some embodiments, the AAV includes a nucleic acid sequence that is at least 93% identical to the nucleic acid sequence of SEQ ID NO: 1. In some embodiments, the AAV includes a nucleic acid sequence that is at least 94% identical to the nucleic acid sequence of SEQ ID NO: 1. In some embodiments, the AAV includes a nucleic acid sequence that is at least 95% identical to the nucleic acid sequence of SEQ ID NO: 1. In some embodiments, the AAV includes a nucleic acid sequence that is at least 96% identical to the nucleic acid sequence of SEQ ID NO: 1. In some embodiments, the AAV includes a nucleic acid sequence that is at least 97% identical to the nucleic acid sequence of SEQ ID NO: 1. In some embodiments, the AAV includes a nucleic acid sequence that is at least 98% identical to the nucleic acid sequence of SEQ ID NO: 1. In some embodiments, the AAV includes a nucleic acid sequence that is at least 99% identical to the nucleic acid sequence of SEQ ID NO: 1. In some embodiments, the AAV includes the nucleic acid sequence of SEQ ID NO: 1.

SEQ ID NO: 1 (Murine Pla2g2f nucleic acid
sequence):
ATGGCAGATGGGGCACAGGCCAACCCCAAAGGGTTCAGGAAGAAGGCGC
TGGTTAAACACTCCACTGGACGGAAGAGCCCAAGCCTCAGGGCCTCTCC
CTCTAAAACCTCCAGGTCCAGCCTGGGTATGAAGAAATTCTTTGCCATC
GCAGTCCTGGCCGGCAGTGTGGTAACCACGGCCCACAGCAGCCTGCTGA
ACCTGAAGTCCATGGTGGAGGCCATCACACACAGAAACTCCATCCTGTC
CTTTGTGGGCTACGGCTGCTACTGCGGGCTGGGGGGACGCGGCCATCCC
ATGGATGAGGTAGACTGGTGCTGCCATGCCCACGACTGTTGCTATGAGA
AGCTCTTTGAGCAGGGCTGCCGCCCCTACGTGGACCACTATGACCACAG
GATCGAGAATGGCACCATGATTGTCTGCACTGAGCTCAATGAGACGGAG
TGTGACAAGCAGACATGCGAGTGTGACAAGAGCCTGACTCTGTGCCTCA
AGGATCACCCATACAGGAACAAGTACCGAGGCTACTTCAACGTCTACTG
CCAGGGCCCCACACCCAACTGCAGCATCTATGACCCGTACCCAGAGGAA
GTCACCTGTGGGCATGGCCTCCCTGCGACCCCTGTCTCAACCTAG

In some embodiments, the AAV includes a nucleic acid sequence that is at least 70% (e.g., at least 75%, 80%, 85%, 86%, 87%, 88%, 89%, 90%, 91%, 92%, 93%, 94%, 95%, 96%, 97%, 98%, 99%, or 99.9%) identical to the nucleic acid sequence of SEQ ID NO: 2. For example, in some embodiments, the AAV includes a nucleic acid sequence that is at least 75% identical to the nucleic acid sequence of SEQ ID NO: 2. In some embodiments, the AAV includes a nucleic acid sequence that is at least 80% identical to the nucleic acid sequence of SEQ ID NO: 2. In some embodiments, the AAV includes a nucleic acid sequence that is at least 85% identical to the nucleic acid sequence of SEQ ID NO: 2. In some embodiments, the AAV includes a nucleic acid sequence that is at least 86% identical to the nucleic acid sequence of SEQ ID NO: 2. In some embodiments, the AAV includes a nucleic acid sequence that is at least 87% identical to the nucleic acid sequence of SEQ ID NO: 2. In some embodiments, the AAV includes a nucleic acid sequence that is at least 88% identical to the nucleic acid sequence of SEQ ID NO: 2. In some embodiments, the AAV includes a nucleic acid sequence that is at least 89% identical to the nucleic acid sequence of SEQ ID NO: 2. In some embodiments, the AAV includes a nucleic acid sequence that is at least 90% identical to the nucleic acid sequence of SEQ ID NO: 2. In some embodiments, the AAV includes a nucleic acid sequence that is at least 91% identical to the nucleic acid sequence of SEQ ID NO: 2. In some embodiments, the AAV includes a nucleic acid sequence that is at least 92% identical to the nucleic acid sequence of SEQ ID NO: 2. In some embodiments, the AAV includes a nucleic acid sequence that is at least 93% identical to the nucleic acid sequence of SEQ ID NO: 2. In some embodiments, the AAV includes a nucleic acid sequence that is at least 94% identical to the nucleic acid sequence of SEQ ID NO: 2. In some embodiments, the AAV includes a nucleic acid sequence that is at least 95% identical to the nucleic acid sequence of SEQ ID NO: 2. In some embodiments, the AAV includes a nucleic acid sequence that is at least 96% identical to the nucleic acid sequence of SEQ ID NO: 2. In some embodiments, the AAV includes a nucleic acid sequence that is at least 97% identical to the nucleic acid sequence of SEQ ID NO: 2. In some embodiments, the AAV includes a nucleic acid sequence that is at least 98% identical to the nucleic acid sequence of SEQ ID NO: 2. In some embodiments, the AAV includes a nucleic acid sequence that is at least 99% identical to the nucleic acid sequence of SEQ ID NO: 2. In some embodiments, the AAV includes the nucleic acid sequence of SEQ ID NO: 2.

SEQ ID NO: 2 (Human Pla2g2f nucleic acid sequence;
www.ncbi.nlm.nih.gov/nucleotide/NM 022819.3):
ATGGCAGATGGGGCAAAGGCCAACCCCAAAGGGTTCAAAAAGAAGGTGC
TGGATAGATGCTTCTCTGGGTGGAGGGGCCCACGCTTCGGGGCCTCCTG
TCCTTCAAGAACCTCCAGGTCTAGCCTGGGTATGAAGAAGTTCTTCACC
GTGGCCATCCTTGCTGGCAGCGTTCTGTCCACAGCTCACGGCAGCCTGC
TCAACCTGAAGGCCATGGTGGAGGCCGTCACAGGGAGGAGCGCCATCCT
GTCCTTCGTGGGCTACGGTTGCTACTGTGGGCTGGGGGGCCGTGGCCAG
CCCAAGGATGAGGTGGACTGGTGCTGCCACGCCCACGACTGCTGCTACC
AGGAACTCTTTGACCAAGGCTGTCACCCCTATGTGGACCACTATGATCA
CACCATCGAGAACAACACTGAGATAGTCTGCAGTGACCTCAACAAGACA
GAGTGTGACAAGCAGACATGCATGTGTGACAAGAACATGGTTCTGTGCC
TCATGAACCAGACGTACCGAGAGGAGTACCGTGGCTTCCTCAATGTCTA
CTGCCAGGGCCCCACGCCCAACTGCAGCATCTATGAACCGCCCCCTGAG
GAGGTCACCTGCAGTCACCAATCCCCAGCGCCCCCCGCCCCTCCCTAG

In some embodiments, the AAV includes an amino acid sequence that is at least 70% (e.g., at least 75%, 80%, 85%, 86%, 87%, 88%, 89%, 90%, 91%, 92%, 93%, 94%, 95%, 96%, 97%, 98%, 99%, or 99.9%) identical to the amino acid sequence of SEQ ID NO: 3. For example, in some embodiments, the AAV includes an amino acid sequence that is at least 70% identical to the amino acid sequence of SEQ ID NO: 3. In some embodiments, the AAV includes an amino acid sequence that is at least 75% identical to the amino acid sequence of SEQ ID NO: 3. In some embodiments, the AAV includes an amino acid sequence that is at least 80% identical to the amino acid sequence of SEQ ID NO: 3. In some embodiments, the AAV includes an amino acid sequence that is at least 85% identical to the amino acid sequence of SEQ ID NO: 3. In some embodiments, the AAV includes an amino acid sequence that is at least 86% identical to the amino acid sequence of SEQ ID NO: 3. In some embodiments, the AAV includes an amino acid sequence that is at least 87% identical to the amino acid sequence of SEQ ID NO: 3. In some embodiments, the AAV includes an amino acid sequence that is at least 88% identical to the amino acid sequence of SEQ ID NO: 3. In some embodiments, the AAV includes an amino acid sequence that is at least 89% identical to the amino acid sequence of SEQ ID NO: 3. In some embodiments, the AAV includes an amino acid sequence that is at least 90% identical to the amino acid sequence of SEQ ID NO: 3. In some embodiments, the AAV includes an amino acid sequence that is at least 91% identical to the amino acid sequence of SEQ ID NO: 3. In some embodiments, the AAV includes an amino acid sequence that is at least 92% identical to the amino acid sequence of SEQ ID NO: 3. In some embodiments, the AAV includes an amino acid sequence that is at least 93% identical to the amino acid sequence of SEQ ID NO: 3. In some embodiments, the AAV includes an amino acid sequence that is at least 94% identical to the amino acid sequence of SEQ ID NO: 3. In some embodiments, the AAV includes an amino acid sequence that is at least 95% identical to the amino acid sequence of SEQ ID NO: 3. In some embodiments, the AAV includes an amino acid sequence that is at least 96% identical to the amino acid sequence of SEQ ID NO: 3. In some embodiments, the AAV includes an amino acid sequence that is at least 97% identical to the amino acid sequence of SEQ ID NO: 3. In some embodiments, the AAV includes a nucleic acid sequence that is at least 98% identical to the amino acid sequence of SEQ ID NO: 3. In some embodiments, the AAV includes an amino acid sequence that is at least 99% identical to the amino acid sequence of SEQ ID NO: 3. In some embodiments, the AAV includes the amino acid sequence of SEQ ID NO: 3.

(Murine Pla2g2f amino acid sequence)
SEQ ID NO: 3
MADGAQANPKGFRKKALVKHSTGRKSPSLRASPSKTSRSSLGMKKFFAI
AVLAGSVVTTAHSSLLNLKSMVEAITHRNSILSFVGYGCYCGLGGRGHP
MDEVDWCCHAHDCCYEKLFEQGCRPYVDHYDHRIENGTMIVCTELNETE
CDKQTCECDKSLTLCLKDHPYRNKYRGYFNVYCQGPTPNCSIYDPYPEE
VTCGHGLPATPVST

In some embodiments, the AAV includes an amino acid sequence that is at least 70% (e.g., at least 75%, 80%, 85%, 86%, 87%, 88%, 89%, 90%, 91%, 92%, 93%, 94%, 95%, 96%, 97%, 98%, 99%, or 99.9%) identical to the amino acid sequence of SEQ ID NO: 4. For example, in some embodiments, the AAV includes an amino acid sequence that is at least 70% identical to the amino acid sequence of SEQ ID NO: 4. In some embodiments, the AAV includes an amino acid sequence that is at least 75% identical to the amino acid sequence of SEQ ID NO: 4. In some embodiments, the AAV includes an amino acid sequence that is at least 80% identical to the amino acid sequence of SEQ ID NO: 4. In some embodiments, the AAV includes an amino acid sequence that is at least 85% identical to the amino acid sequence of SEQ ID NO: 4. In some embodiments, the AAV includes an amino acid sequence that is at least 86% identical to the amino acid sequence of SEQ ID NO: 4. In some embodiments, the AAV includes an amino acid sequence that is at least 87% identical to the amino acid sequence of SEQ ID NO: 4. In some embodiments, the AAV includes an amino acid sequence that is at least 88% identical to the amino acid sequence of SEQ ID NO: 4. In some embodiments, the AAV includes an amino acid sequence that is at least 89% identical to the amino acid sequence of SEQ ID NO: 4. In some embodiments, the AAV includes an amino acid sequence that is at least 90% identical to the amino acid sequence of SEQ ID NO: 4. In some embodiments, the AAV includes an amino acid sequence that is at least 91% identical to the amino acid sequence of SEQ ID NO: 4. In some embodiments, the AAV includes an amino acid sequence that is at least 92% identical to the amino acid sequence of SEQ ID NO: 4. In some embodiments, the AAV includes an amino acid sequence that is at least 93% identical to the amino acid sequence of SEQ ID NO: 4. In some embodiments, the AAV includes an amino acid sequence that is at least 94% identical to the amino acid sequence of SEQ ID NO: 4. In some embodiments, the AAV includes an amino acid sequence that is at least 95% identical to the amino acid sequence of SEQ ID NO: 4. In some embodiments, the AAV includes an amino acid sequence that is at least 96% identical to the amino acid sequence of SEQ ID NO: 4. In some embodiments, the AAV includes an amino acid sequence that is at least 97% identical to the amino acid sequence of SEQ ID NO: 4. In some embodiments, the AAV includes a nucleic acid sequence that is at least 98% identical to the amino acid sequence of SEQ ID NO: 4. In some embodiments, the AAV includes an amino acid sequence that is at least 99% identical to the amino acid sequence of SEQ ID NO: 4. In some embodiments, the AAV includes the amino acid sequence of SEQ ID NO: 4.

(Human Pla2g2rf amino acid sequence;
www.ncbi.nlm.nih.gov/protein/145553989)
SEQ ID NO: 4
MADGAKANPKGFKKKVLDRCFSGWRGPRFGASCPSRTSRSSLGMKKFFT
VAILAGSVLSTAHGSLLNLKAMVEAVTGRSAILSFVGYGCYCGLGGRGQ
PKDEVDWCCHAHDCCYQELFDQGCHPYVDHYDHTIENNTEIVCSDLNKT
ECDKQTCMCDKNMVLCLMNQTYREEYRGFLNVYCQGPTPNCSIYEPPPE
EVTCSHQSPAPPAPP

### IC. Anti-Sense Oligonucleotides (ASOs)

The compositions and methods described herein may be used in combination with one or more ASOs to enhance Pla2g2f expression in target cells. ASO-mediated increase in protein expression can occur at the level of splicing or translation. ASOs that target three different types of non-productive splicing events have been identified in a screen in (Lim, et al., (2020) Nat Commun 11, 3501). Specifically, ASOs targeting the following gene targets (PCCA, SYNGAP1, CD274, and SCN1A) are known to non-productive splicing events and led to an inversely correlated increase of productive mRNA. These ASOs are anticipated to advantageously enhance Pla2g2f expression in target cells when administered in combination with the compositions and methods described herein. Other ASOs identified in the screen are also considered to be exemplary ASOs for use in combination with the compositions and methods of the disclosure. Additionally, exemplary ASOs may include those that bind to mRNA sequences in upstream open reading frames to specifically increase the amounts of protein translated from a downstream primary open reading frame, which are known in the art.

ID. Endonuclease-Deficient (“Dead”) Cas9 (dCas9) System

The compositions and methods described herein may be used in combination with dCas9 systems that contain sgRNAs encoding Pla2g2f to enhance Pla2g2f expression in target cells. It is known in the art that dCas9 system can be used to modulate transcription of a gene of interest. When the deactivated protein is fused to a transcriptional activator domain, such as VP64, dCas9 binds to the sequence complementary to its sgRNA, and the activator domain recruits RNA Polymerase II to begin or enhance transcription of the gene of interest, e.g., a gene encoding Pla2g2f.

### II. Pharmaceutical Carriers for Enhanced Delivery

In some embodiments, the compositions described herein are in the form of pharmaceutical carriers, including, but not limited to, nanoparticles (e.g., lipid nanoparticles (LNPs), and exosomes. Methods for producing carriers are known in the art.

LNPs possess a lipid core matrix that can solubilize lipophilic molecules. The lipid core is stabilized by surfactants (emulsifiers). The term lipid is used here in a broader sense and includes triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate). The core lipids can be fatty acids, acylglycerols, waxes, and mixtures of these surfactants. Biological membrane lipids such as phospholipids, sphingomyelins, bile salts (sodium taurocholate), and sterols (cholesterol) are utilized as stabilizers. Emulsifiers may be used to stabilize the lipid dispersion.

LNPs can be produced (e.g., allowed to self-assemble, e.g., spontaneously) by injecting a lower alkanol solution containing lipids into an aqueous solution. Various lipids can be used to achieve desired properties, such as size, surface charge, and capacity for encapsulants. Such properties can also be influenced by the composition of the aqueous solution. LNPs of the invention can encapsulate a wide range of hydrophilic molecules, e.g., oligonucleotides. Variations in lipid nanoparticle size can be affected by controlling process parameters. In particular, the rate at which the lower alkanol solution is injected into the aqueous solution is inversely related to the resulting lipid nanoparticle size. Similarly, minimizing variance in the rate of injection will minimize variance in lipid nanoparticle size, yielding homogeneous suspensions of LNPs, e.g., within a single batch or among multiple batches. A precise rate of injection can be attained, e.g., through a servo pump.

Typically, the LNPs are liposomes with a lipid bilayer surrounding an aqueous interior. Liposomes can be of different sizes. Liposome design may include ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as the pharmaceutical formulation to be encapsulated, the liposomal ingredients, the nature of the medium in which the lipid vesicles are dispersed (e.g., osmolality or pH), the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, the compositions described herein are associated with (e.g., encapsulated by) a LNP. In some embodiments, the LNP-associated composition is administered to a subject (e.g., a human subject having a disease or condition). Exemplary LNPs include spheres, ellipsoids, rods, and discs. Other LNPs that can be used to encapsulate oligonucleotides encoding viral vectors and/or transgenes are well known in the art.

Other pharmaceutical carriers include exosomes. Exosomes are small membranous vesicles secreted by most cell types. These vesicles are generally 50-100 nm in diameter. Exosomes have been shown to mediate cell-cell transmission in, for example, immune cells or tumor cells, by fusing with the target-cell membrane and releasing their cargo into the target cell. Therefore, exosomes present an efficacious mode of delivery for encapsulated loads, such as oligonucleotides, e.g., oligonucleotides encoding viral vectors.

In some embodiments, the compositions described herein are encapsulated by an exosome. Exemplary exosomes of the disclosure include those between 50-100 nm (e.g., between 60-90 nm, 70-80 nm, or 75 nm). For example, in some embodiments, the compositions described herein are encapsulated by an exosome that is between 50-100 nm in size. In some embodiments, the compositions described herein are encapsulated by an exosome that is between 60-90 nm in size. In some embodiments, the compositions described herein are encapsulated by an exosome that is between 70-80 nm in size. In some embodiments, the compositions described herein are encapsulated by an exosome that is 75 nm in size.

### Methods of Detecting RNA Transcript Expression

The expression level of a RNA transcript, such as a Pla2g2f mRNA transcript, can be ascertained, for example, by a variety of nucleic acid detection techniques. Additionally or alternatively, RNA transcript expression can be inferred by evaluating the concentration or relative abundance of an encoded protein produced by translation of the RNA transcript. Protein concentrations can also be assessed, for example, using functional assays. Using these techniques, an increase in the concentration of Pla2g2f mRNA transcripts in response to the compositions and methods described herein can be observed, while monitoring the expression of the encoded protein. The sections that follow describe exemplary techniques that can be used to measure the expression level of an RNA transcript and its downstream protein product. RNA transcript expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of RNAs.

### Nucleic Acid Detection

Nucleic acid-based methods for detection of DNA or RNA transcript expression include imaging-based techniques (e.g., Northern blotting or Southern blotting), which may be used in conjunction with cells obtained from a patient following administration of, for example, a vector encoding a transgene encoding Pla2g2f or a composition containing such a construct. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, NY 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

RNA detection techniques that may be used in conjunction with the compositions and methods described herein to evaluate the efficacy of administration of any of the compositions described herein further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For example, transgene expression at the mRNA level may be determined using RNA-Seq (e.g., as described in Mortazavi et al., Nat. Methods 5:621-628 (2008), the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology®). Then, the cDNA is converted into a molecular library for sequencing by addition of sequence adapters for each library (e.g., from Illumina®/Solexa), and the resulting 50-100 nucleotide reads are mapped onto the genome.

RNA expression levels may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and includes arrays fabricated by direct synthesis of oligonucleotides on a glass surface. Other systems may be used as known to one skilled in the art.

Amplification-based assays also can be used to measure the expression level of a particular RNA transcript, such as a Pla2g2f mRNA transcript. In such assays, the nucleic acid sequence of the transcript acts as a template in an amplification reaction (for example, PCR, such as qPCR). In a quantitative amplification, the amount of amplification product is proportional to the amount of template in the original sample. Comparison to appropriate controls provides a measure of the expression level of the transcript of interest, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of RNA transcript expression as described herein can be determined, for example, by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Expression of an RNA construct may also be inferred by analyzing expression of the protein encoded by the construct. Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize transgene expression in a cell from a patient (e.g., a human patient) following delivery of the transgene. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Pharmaceutical Compositions

The nucleic acid molecules described herein can be formulated into pharmaceutical compositions for administration to a patient, such as a human patient having neurodegeneration, such as a patient diagnosed with a neurodegenerative disorder (e.g., AD, FTD, PD, or ALS) or displaying one or more symptoms of neurodegeneration (e.g., cognitive decline or neuroinflammation), in a biologically compatible form suitable for administration in vivo. A pharmaceutical composition containing, for example, a nucleic acid molecule including a transgene encoding Pla2g2f, as described herein, typically includes a pharmaceutically acceptable diluent or carrier. A pharmaceutical composition may include (e.g., consist of), e.g., a sterile saline solution and a nucleic acid. The sterile saline is typically a pharmaceutical grade saline. A pharmaceutical composition may include (e.g., consist of), e.g., sterile water and a nucleic acid. The sterile water is typically a pharmaceutical grade water. A pharmaceutical composition may include (e.g., consist of), e.g., phosphate-buffered saline (PBS) and a nucleic acid. The sterile PBS is typically a pharmaceutical grade PBS.

In certain embodiments, pharmaceutical compositions include a plurality of the transgenes and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, nucleic acid molecules may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions including a transgene encoding Pla2g2f encompass any pharmaceutically acceptable salts of the inhibitor, esters of the inhibitor, or salts of such esters. In certain embodiments, pharmaceutical compositions including a transgene encoding a nucleic acid molecule, upon administration to a subject (e.g., a human), are capable of providing (directly or indirectly) the biologically active metabolite or residue thereof. Accordingly, for example, the disclosure is also drawn to pharmaceutically acceptable salts of inhibitors, prodrugs, pharmaceutically acceptable salts of such prodrugs, and other bioequivalents. Suitable pharmaceutically acceptable salts include, but are not limited to, sodium and potassium salts. In certain embodiments, prodrugs include one or more conjugate group attached to a nucleic acid molecule, wherein the conjugate group is cleaved by endogenous nucleases within the body.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used. In certain embodiments, pharmaceutical compositions include one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intraocular (e.g., intravitreal), intravenous, subcutaneous, intramuscular, intrathecal, intracerebroventricular, etc.) either systemically or to a particular tissue. In certain of such embodiments, a pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

### Routes of Administration

Viral vectors, such as AAV vectors, lentiviral vectors, and others described herein, containing a transgene encoding Pla2g2f described herein may be administered to a patient (e.g., a human patient) by a variety of routes of administration. The route of administration may vary, for example, with the onset and severity of disease, and may include, e.g., intravenous, intrathecal, intracerebroventricular, intraparenchymal, intracisternal, intradermal, transdermal, parenteral, intramuscular, intranasal, subcutaneous, percutaneous, intratracheal, intraperitoneal, intraarterial, intravascular, inhalation, perfusion, lavage, and oral administration. Intravascular administration may include delivery into the vasculature of a patient. In some embodiments, the administration is into a vessel considered to be a vein (intravenous), and in some administration, the administration is into a vessel considered to be an artery (intraarterial). Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Treatment regimens may vary, and often depend on disease severity and the age, weight, and sex of the patient. Treatment may include administration of vectors (e.g., viral vectors) or other agents (e.g., nanoparticles (e.g., LNPs) or exosomes) described herein as useful for the introduction of a transgene into a target cell.

In one embodiment, a viral vector is administered by intraperitoneal administration.

### Combination Therapies

A nucleic acid molecule described herein can be administered in combination with one or more additional therapeutic agents for treatment of a patient having neurodegeneration, such as a human patient diagnosed with or displaying one or more symptoms of neurodegeneration (e.g., cognitive decline). The one or more additional therapeutic agents may include a corticosteroid (e.g., bethamethasone, prednisolone, triamcinolone, methylprednisolone, dexamethasone, hydrocortisone, cortisone, ethamethasoneb, prednisone, prednisolone, triamcinolone, dexamethasone, or fludrocortisone) or an immunosuppressive drug (e.g., pomalidomide, methotrexate, azathioprine, lenalidomide, azathioprine, or thalidomide), or a combination thereof.

### Kits

The compositions described herein can be provided in a kit for use in enhancing metabolism or treating a patient diagnosed with or displaying one or more symptoms of neurodegeneration (e.g., declining cognitive function). In some embodiments, the kit may include a viral vector as described herein. The kit can include a package insert that instructs a user of the kit, such as a physician, to perform any one of the methods described herein. The kit may optionally include a syringe or other device for administering the composition. In some embodiments, the kit may include one or more additional therapeutic agents.

### Examples

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Identification of a Novel Aging-Induced Neuronally Derived Secreted Signal that Activates Neural Stem Cells and Decreases Neuroinflammation (Inflammatory Microglia and Reactive Astrocytes) to Confer Resilience to Synapse Loss and Cognitive Decline in Aging and Alzheimer's Disease (AD)

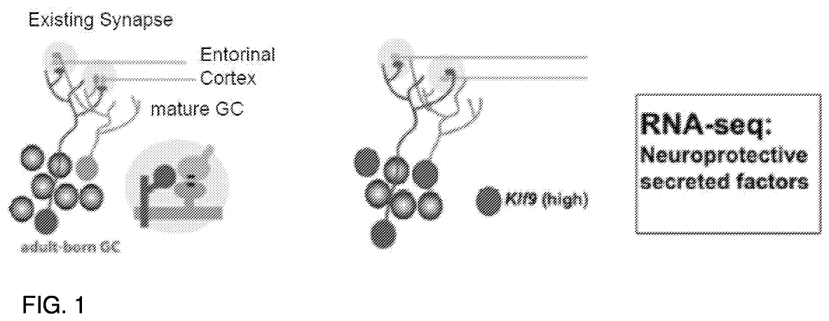

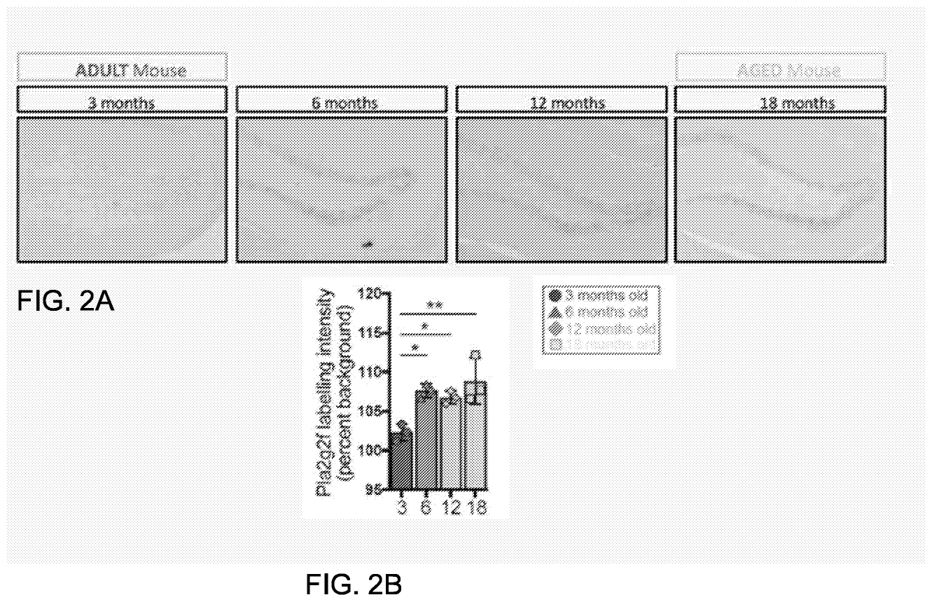

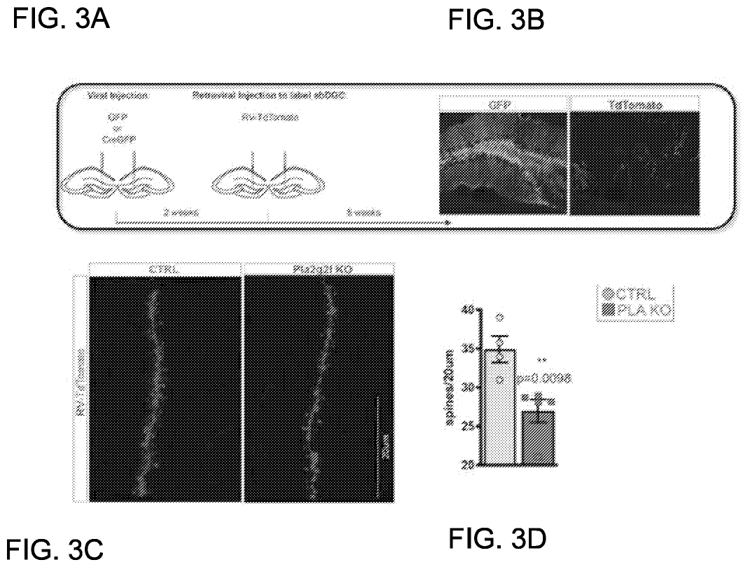

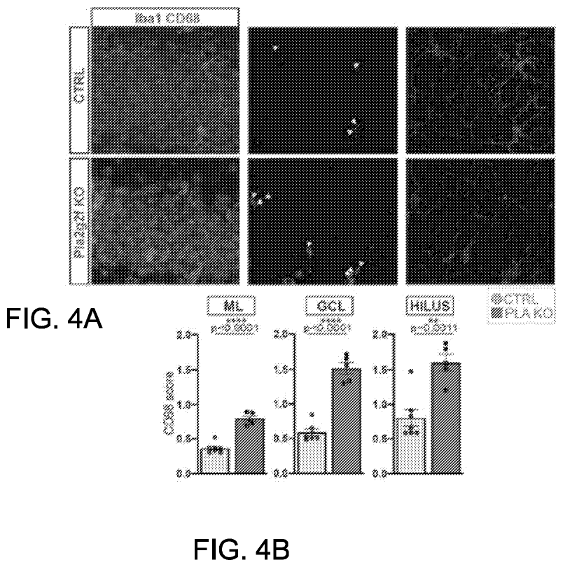

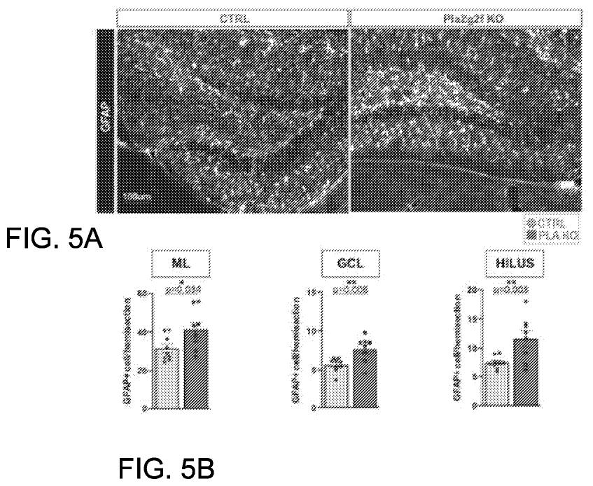

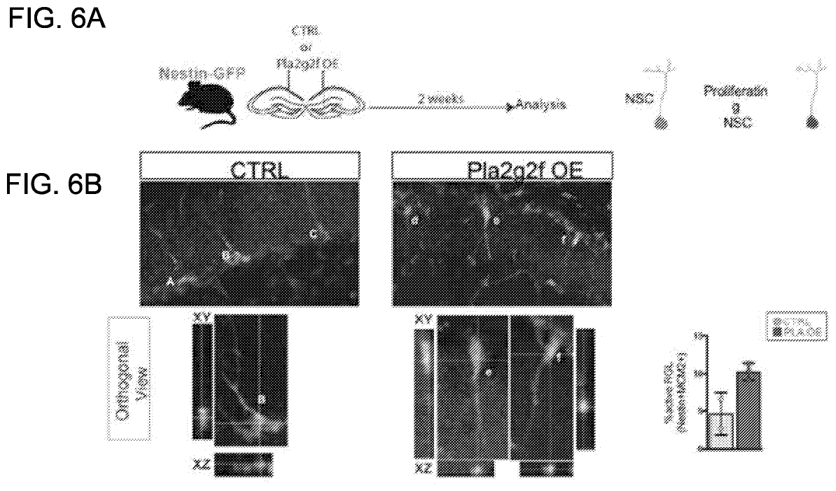

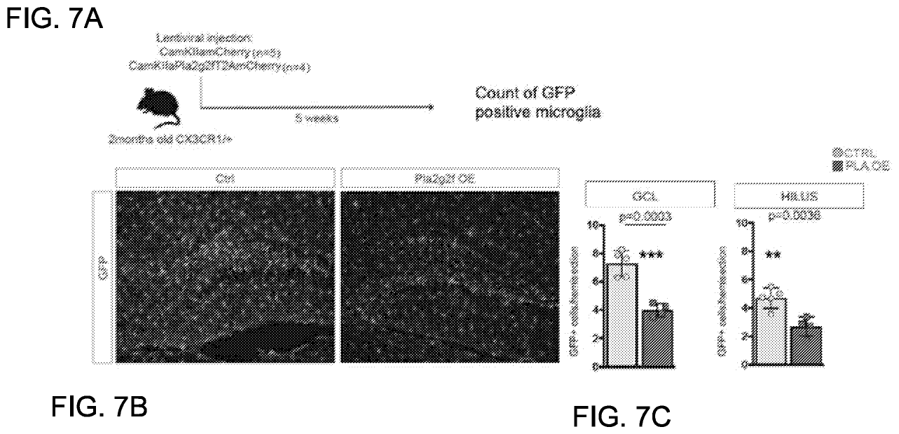

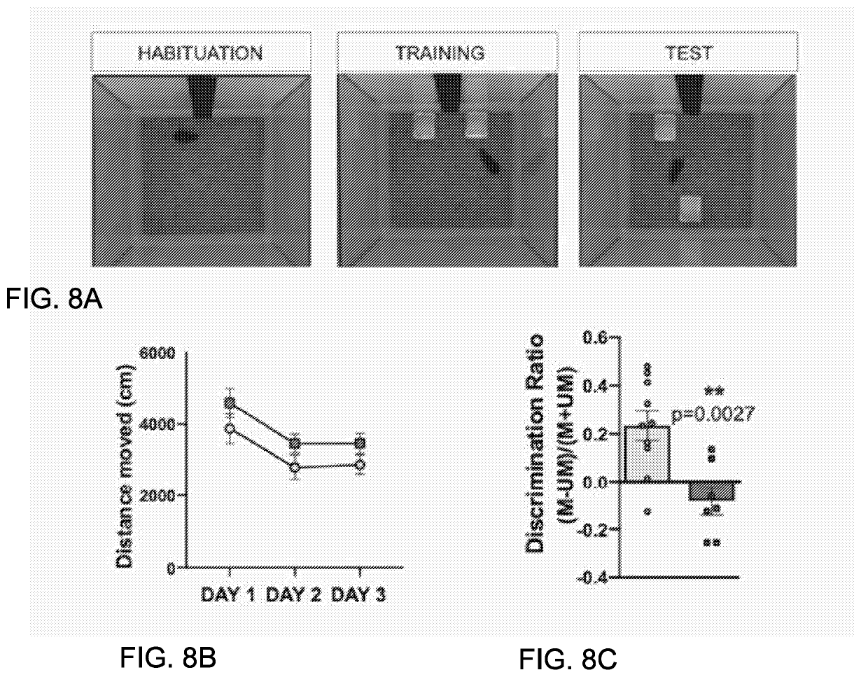

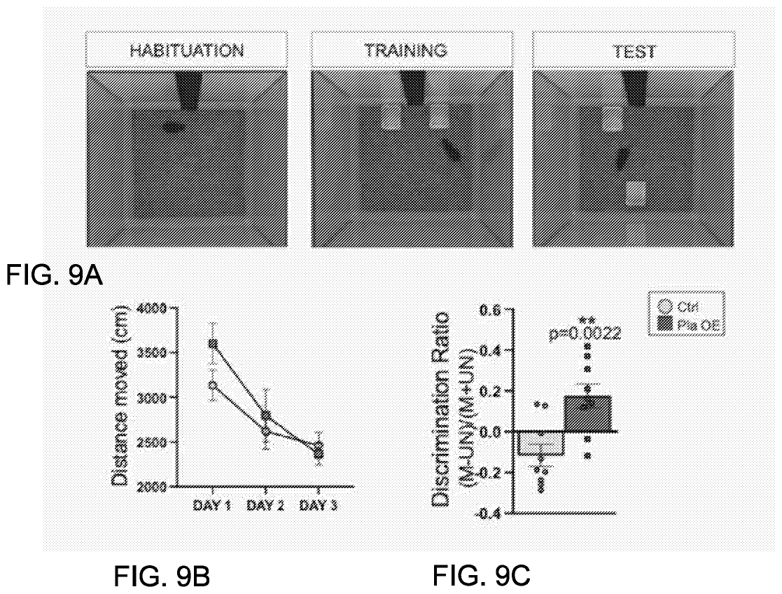

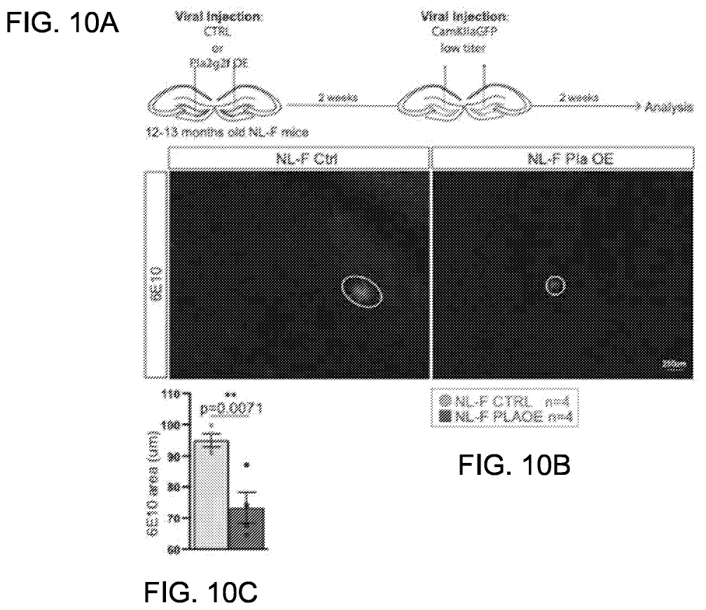

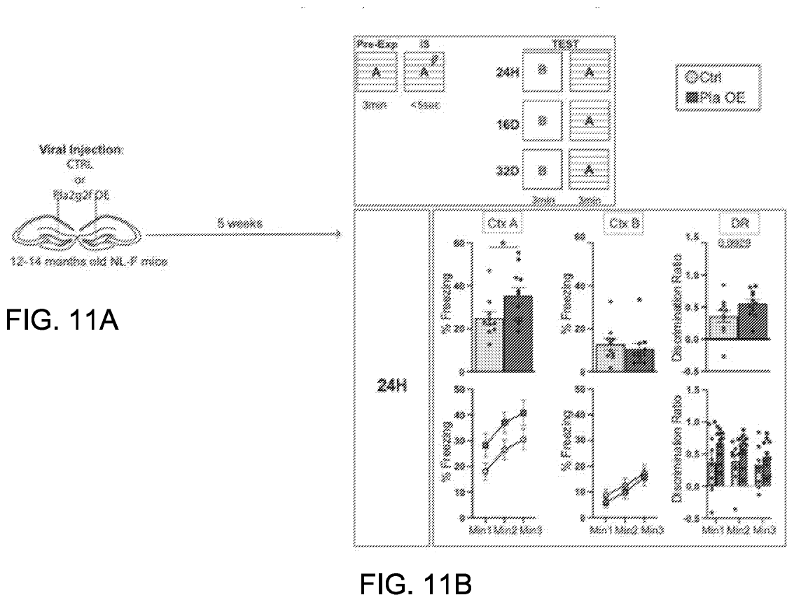

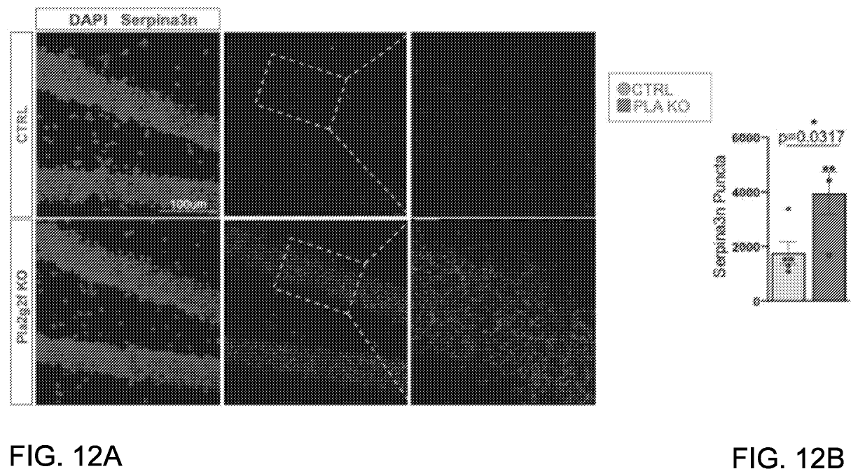

A genetic screen for neuronal derived resilience or compensatory factors in a genetic mouse model of inducible excitatory synapse loss was performed in order to identify dentate granule cell (DGC) derived pro-neurogenic secreted factors (FIG. 1). Pla2g2f was identified as one of the most highly upregulated genes following dendritic spine elimination. In situ hybridization data indicates that hippocampal Pla2g2f expression is negligible in adulthood but steadily increases exclusively in the dentate gyrus (DG) during aging (FIG. 2). Conversely, viral deletion of Pla2g2fin DGCs of conditional mutant mice in middle age, when Pla2g2f levels are normally elevated, resulted in synapse loss (FIG. 3) and an increase in microglia (FIG. 4) and reactive astrocytes (FIG. 5). Furthermore, viral and genetic gain of function approaches to overexpress Pla2g2f in DGCs of aged mice increased neuronal stem cell (NSC) activation (FIG. 6) and reduced microglia density (FIG. 7), suggesting a role for Pla2g2f in reducing neuroinflammation. Consistent with these changes induced by bidirectional regulation of Pla2g2f levels, deletion of Pla2g2f in middle-aged mice induced premature aging-associated memory impairments (FIG. 8), whereas Pla2g2f overexpression protected aged mice from memory loss (FIG. 9). Subsequently, viral overexpression of Pla2g2f in a next-generation murine model of AD (aged APP-NLF knock-in mice) reduced amyloid burden (FIG. 10) and importantly, protected against memory loss, as seen by improved memory retrieval in a contextual fear conditioning behavioral test (FIG. 11). Additionally, deletion of Pla2g2f resulted in an increase in Serpina3n (FIG. 12), a secreted serine protease inhibitor that is associated with amyloid deposits and elevated in the CSF of AD individuals.

### Example 2. Materials and Methods

### Mouse Lines

Pla2g2f conditional KO (Pla2g2f cKO) animals were generated as previously described (Yamamoto et al. (2015) The Journal of Experimental Medicine 212, 1901-1919) and kindly donated by Dr. Makoto Murakami and Dr. Kei Yamamoto.

CamKIIaCreERT2 animals were generated as previously described (Volk, et al. (2013) Nature 493, 420-423.) and kindly donated by Dr. Richard L. Huganir.

For the viral deletion of Pla2g2f middle-aged animals were used (injection of viral vectors in 9-month-old mice).

For genetic deletion of Pla2g2f, 12-month-old CamKIIaCreERT2/+Pla2g2f cKO animals were intraperitoneally injected for 5 consecutive days, once a day with 100 mg/kg dose of either vehicle (corn oil) or tamoxifen (Sigma #T5648).

B6.129P2(Cg)-Cx3cr1^{tm1Litt}/J (referred to as CX3CR1-GFP) were purchased from Jackson Laboratory (www.jax.org/strain/005582).

App NL-F/NL-F (referred to as NL-F) mice were generated as previously described (Saito, et al., (2014) Nat Neurosci 17, 661-663 (2014)) and kindly provided to us by Dr. Takaomi C. Saido.

### Viral Vectors

Viral vectors were purchased from Vector Builder.

For viral deletion of Pla2g2f, the following viral plasmids were used:
CamKIIa-GFP (Vector Name: pLV[Exp]-CaMKIIa_short>EGFP)
CamKIIa-Cre-T2A-GFP (Vector Name:pLV[Exp]-CaMKIIa_short>Cre(ns):T2A:EGFP)

For viral overexpression of Pla2g2f, the following viral plasmids were used:
CamKIIa-mCherry (Vector Name: pLV[Exp]-{CamKIIa}>mCherry)
CamKIIa-Pla2g2f-T2A-mCherry (Vector Name: pLV[Exp]-
{CamKIIa}>mPla2g2f[NM_012045.4](ns):T2A:mCherry)

### In Situ Hybridization (ISH)

ISH was performed using Pla2g2f-specific riboprobe generated from the coding sequence region of mouse Pla2g2f (NM_001360875.1) corresponding to nucleotides 252-755.

Color reaction was carried out with nitro blue tetrazolium (NBT)/5-bromo-4-chloro-3-indolyl-phosphate (BCIP). Color reaction times were identical for every group. For quantification, three sections per mouse were analyzed using the mean intensity function in Fiji. All images were captured using the same light intensity and exposure times. The mean intensity of the region of interest (minus mean intensity of a selected background region) was averaged across images for each mouse and each group.

### RNA Scope

Fresh-frozen coronal serial sections (14 um) were collected and RNAscope Multiplex Fluorescent Detection Kit was used to detect Serpina3n signal (Cat No. 430191 Probe Mm Serpina3n Cl).

### Immunohistochemistry

Coronal serial sections (35 μm) were obtained using a Leica cryostat in 6 matched sets. One set of sections was washed three times in PBS with 0.3% Triton X-100 and then blocked in PBS with 0.3% Triton X-100 and 10% NDS (normal donkey serum) for 2 hours at room temperature. Incubation with primary antibodies was carried out O/N at 4° C.

### Antibodies:

Iba1:Wako Rb Chem 019-19741 1:500
GFAP:DAKO Rb Z0334 1:500
GFP: Aves Chicken GFP-1020 1:500
CD68: Biorad Rat MCA1957 1:200
RFP: Rockland Rb 600-401-379 1:200
6E10: Biolegend 80300 Mouse Anti-Ab 1-16 1:500

For 6E10 immunohistochemistry, sections were blocked in in PBS with 0.3% Triton X-100, 5% NDS (normal donkey serum) and 5% BSA (Bovine Albumin Serum).

Fluorescent labeled coupled secondary antibodies (Jackson ImmunoResearch) were used at a final concentration of 1:500 in PBS.

### Confocal Imaging

For spine imaging, confocal z stack images were acquired using a Leica Sp8 confocal microscope (Leica TCS SP8) with 63× oil objective, plus 4× digital zoom to generate high-resolution images. Confocal 9 μm z stacks with 0.3 μm step size were taken centered on the dendritic segment. Z stacks were flattened using the maximum intensity projection, and flattened images were quantified using Fiji. For spine density, spines were counted manually for at least 20 μM of dendritic length per dendrite per mouse.

At least 20 dendritic segments were counted and averaged per mouse per group. For Iba1 and CD68 imaging, confocal z stack images were acquired using Leica Sp8 confocal microscope with 40× water objective. Confocal 25 μm z stacks with 1 μm step size were taken (1024×1024 resolution). Z stacks were flattened using the maximum intensity projection, and flattened images were quantified using Fiji. 3 sections per mouse were analyzed and avaraged. CD68 score in Iba1+cell was assigned as previously described (Hong et al., (2016) Science. 352(6286):712-716).

### Behavioral Assays

Mice were handled for 3 days before starting behavioral experiments.

### Object Location

Mice were introduced in a plastic white arena (41 cm×41 cm) where a black vertical stripe was placed in the middle of the northern wall; the plastic floor of the chamber was covered in white tape, and then a thin even layer of bedding (˜0.5 cm) was added throughout the entire arena. Animals were first habituated to the arena in the absence of any objects across three days, in ten minutes sessions per day.

For training, animals were allowed to freely explore each object (two identical clear Schifferdecker staining jars) in the arena for 15 minutes. For testing, a 10 minutes session was used. During the testing session, one of the objects was displaced. The displaced object was counterbalanced such that the left or right object was equally displaced across groups. Exploring behavior was scored using Noldus Sofware.

### Contextual Fear Conditioning

Mice were allowed to freely explore Context A for 3 minutes in the absence of an aversive stimulus. The conditioning chamber position was randomized for group assignments. 24 hours after context preexposure, animals underwent the same procedures as the day before; however, upon immediate entry into context A, animals received a single 2 seccond, 0.75 mA footshock (Immediate Shock, IS), and were immediately removed. All animals were in the context for no longer than 5 seconds.

Twenty four hours after IS, animals were placed in a novel context (context B) for three minutes. After completion of the context discrimination, animals were placed outside the behavior room, and the room was refitted to be identical to context A; animals were then tested for retrieval in context A (3 minutes) in the absence of any shock. Freezing was scored using FreezeFrame Software.

### Example 3. Treatment of a Human Patient Having Neurodegeneration by Viral Vectors Encoding Pla2g2f

Using the compositions and methods of the disclosure, a human patient having neurodegeneration (e.g., a patient diagnosed with a neurodegenerative disorder or displaying one or more symptoms of neurodegeneration) may be administered a formulation that increases Pla2g2f expression, such as an AAV vector encoding Pla2g2f.

Upon administering the formulation to the patient, the patient may exhibit increased expression of Pla2g2f and may experience an amelioration of one or more symptoms of neurodegeneration, such as declining cognitive function, as measured by improvement in performance on a qualitative test, e.g., by a Montreal Cognitive Assessment test, before and after treatment.

### Example 4. Effects of Pla2g2f Knockout and Overexpression

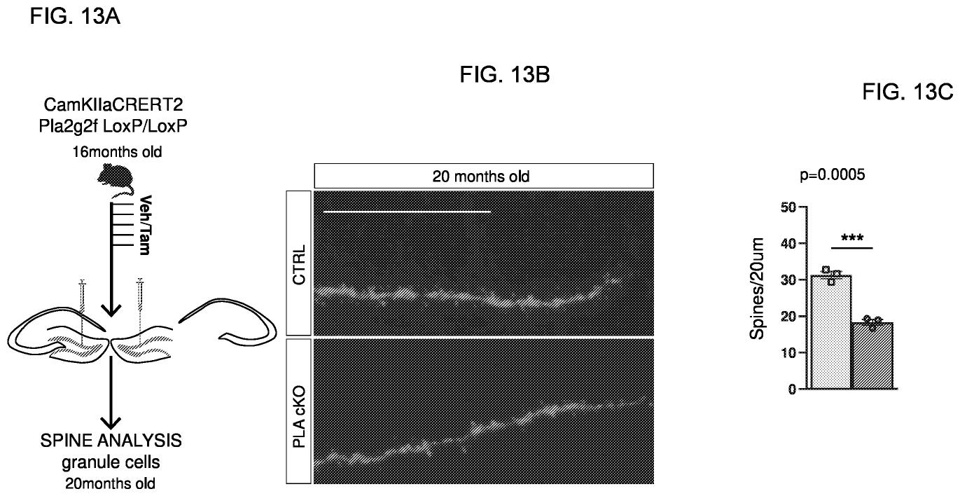

To assess loss of function effects of Pla2g2f during the aging process in vivo, 16 months old CamKIIa-Cre-ERT2 Pla2g2f cKO bigenic mice were intraperitoneally injected with vehicle (ctrl) or tamoxifen (e.g., to induce Pla2g2f deletion) and analyzed (FIG. 13A). CamKIIaCreERT2 animals were generated as previously described (Volk, et al. (2013) Nature 493, 420-423.) and kindly donated by Dr. Richard L. Huganir. Two weeks after injection, both groups of mice were injected in the dentate gyrus with a lentiviral vector expressing mCherry (CamKIIa-mcherry); this labeled mature dentate granule cells. Four months later, the analysis of dendritic spines of dentate granule cells was performed (FIG. 13B and FIG. 13C), revealing that the deletion of Pla2g2f significantly reduced dendritic spine density in mature granule cells.

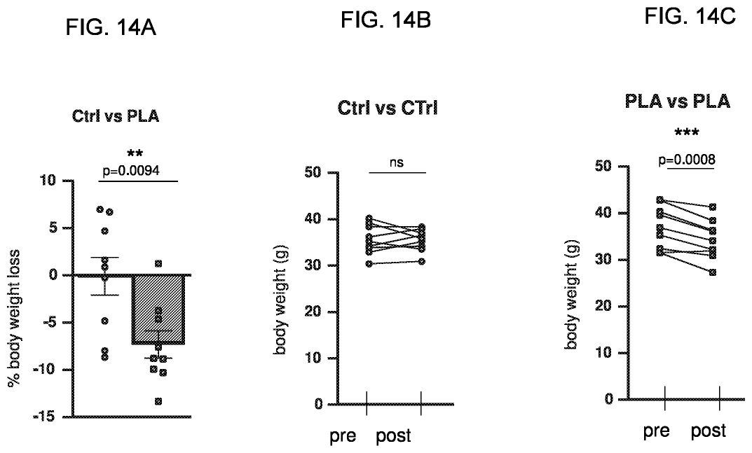

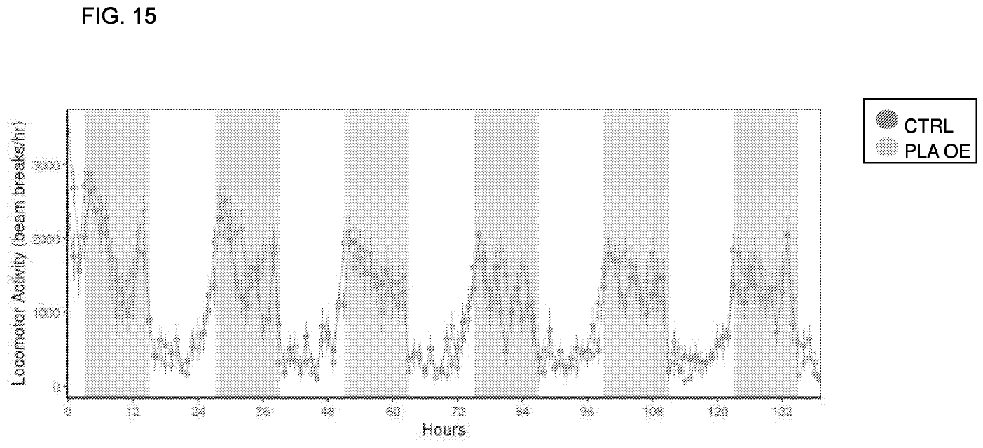

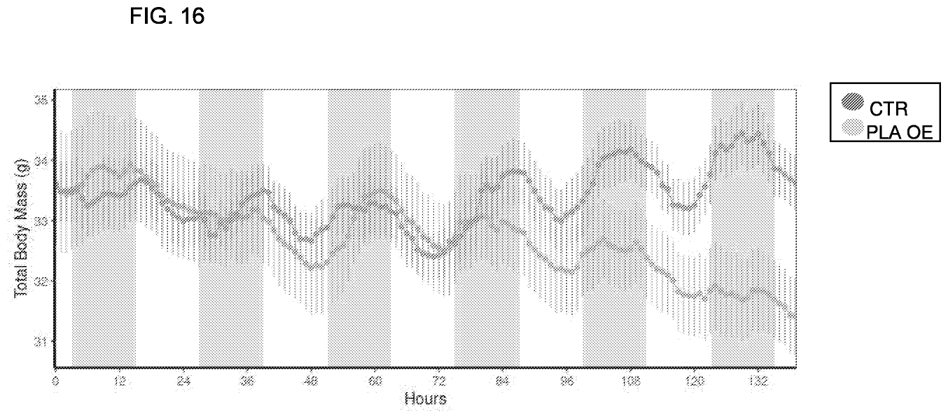

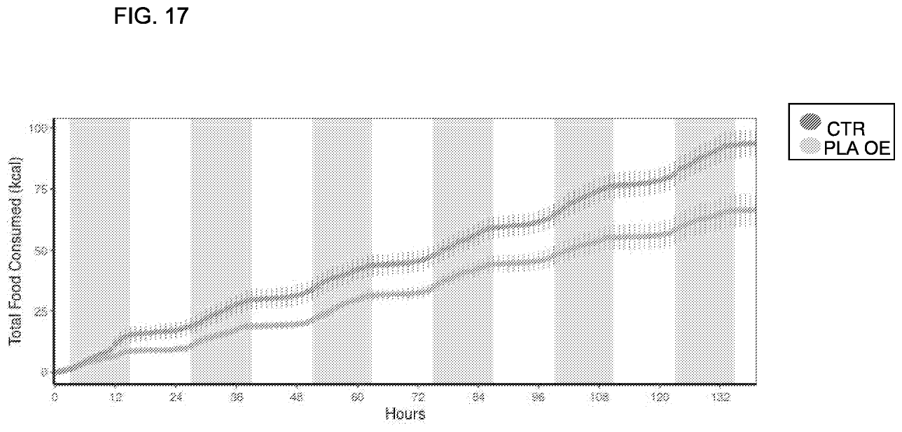

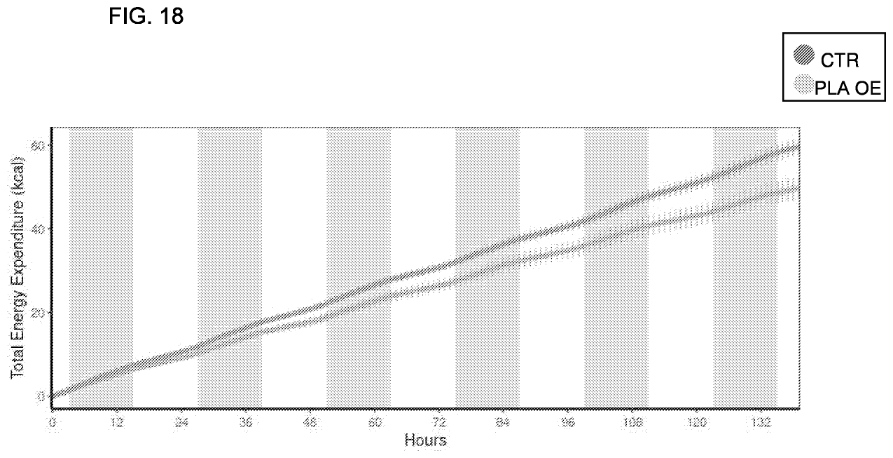

To assess the effects of overexpressing Pla2g2f in vivo, 16 month old C57BL/6 mice were injected with CamKIIa-mCherry (Ctrl) or CamKIIa-Pla2g2f-T2a-mCherry (PLA OE) lentiviruses in the dentate gyrus. Each mouse's body weight (FIG. 14A-FIG. 14C), locomotor activity (FIG. 15), body mass (FIG. 16), food intake (FIG. 17), and energy expenditure (FIG. 18) over time was measured before and after the injection. To measure basal locomotor activity, food intake, body mass, and energy expenditure over time, mice were placed into metabolic cages (Promethion System) for five days. Food and water were provided ad libitum. To measure weight loss, Percent body weight loss was considered the following: [(body weight after injection−body weight before injection)/body weight before injection]×100. Results of this analysis showed that Pla2g2f overexpression resulted in increased locomotor activity (particularly at night but not during the day), reduced body weight/mass, reduced food consumption, and enhanced energy expenditure.

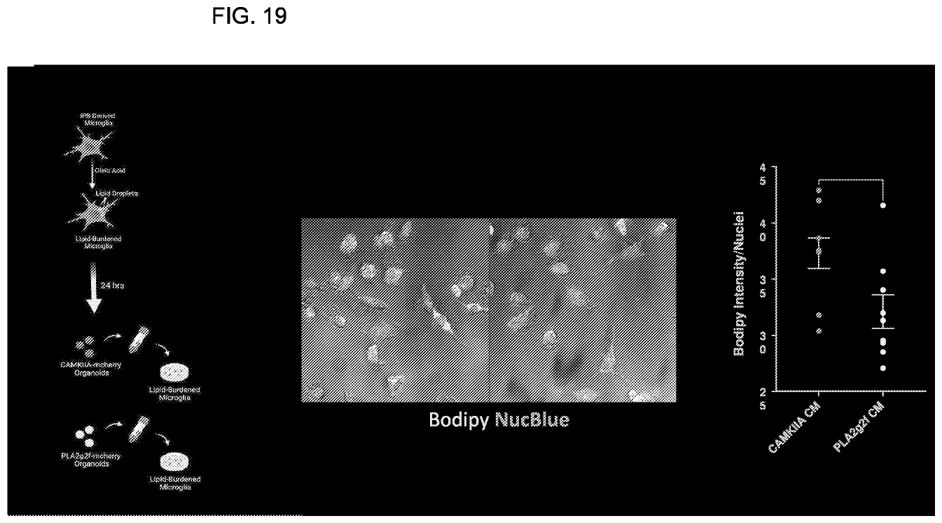

To assess the effects of overexpressing Pla2g2f in vitro, human forebrain organoids were infected with CamKIIa-mCherry (Ctrl) or CamKIIa-Pla2g2f-T2a-mCherry (PLA OE) lentiviruses at DIV (Day in Vitro) 60 (FIG. 19). Human microglia were differentiated from human induced pluripotent stem cells. Microglia were treated with oleic acid for 24 hours to induce lipid droplets formation. Lipid droplets were visualized and quantified using BODIPY staining. Conditioned medium derived from human forebrain organoids with Ctrl or overexpressing Pla2g2f was applied to lipid-burdened human microglia. Results of this analysis suggest that Pla2g2f expression in neurons sends signals to human microglia to reduce lipid burden in said microglia.

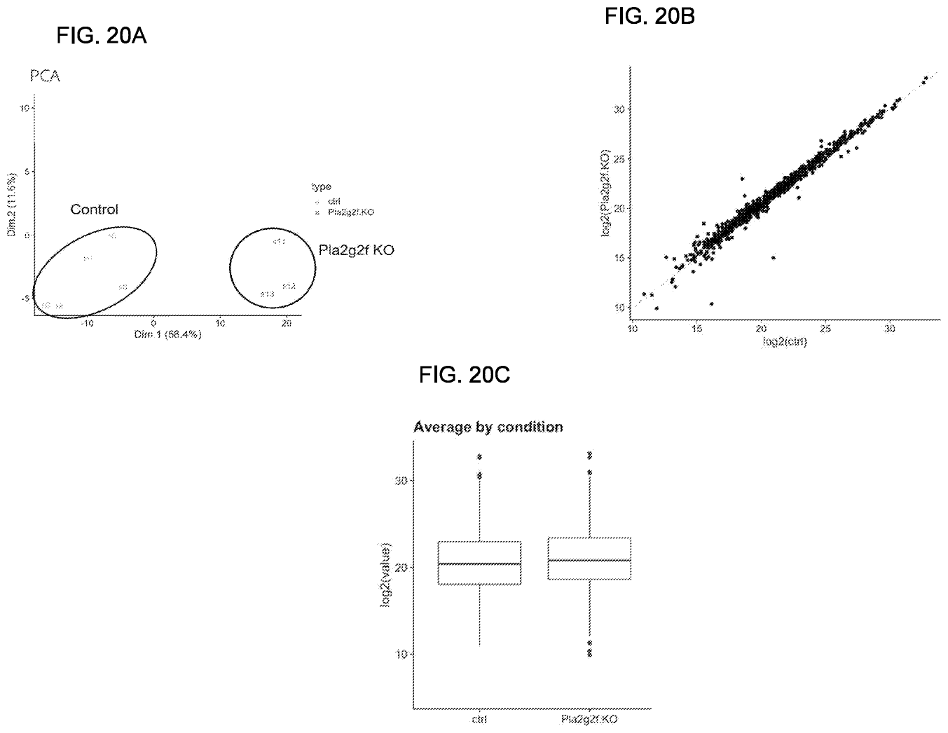

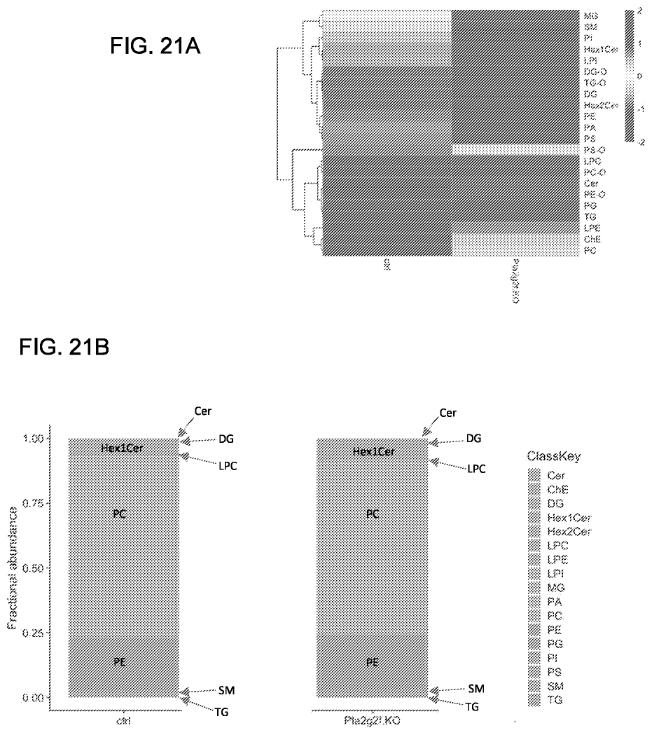

Next, to assess lipid homeostasis, a lipidomics analysis was performed on 16 month old Pla2g2f cKO and C57BL/6 mice injected with CamKIIa Cre-GFP lentivirus. The dentate gyrus was dissected 5 weeks after the lentiviral injection, and the tissue was homogenized in phosphate-buffered saline using Bead Mill Homogenizer (VWR). Subsequently, lipids were extracted according to Folch's Method. The organic phase of each sample, normalized by tissue weight, was then separated using ultra-high performance liquid chromatography coupled to tandem mass spectrometry (UHPLC-MSMS) method. A subsequent PCA analysis (FIG. 20A), distribution analysis of lipid species (FIG. 20B), averaged lipidome abundance per condition (FIG. 20C), and quantification of fractional abundance in lipid species (FIG. 21A and FIG. 21B) of Ctrl and Pla2g2f conditional knockout (cKO) mice showed that that loss of Pla2g2f in aging mice disrupts lipid homeostasis.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A method of enhancing cognitive function, delaying or reducing cognitive decline, or improving memory in a subject, the method comprising administering Pla2g2f to the subject.

2. The method of claim 1, wherein cognitive function is enhanced in the subject.

3. The method of claim 1, wherein cognitive decline is delayed or reduced in the subject.

4. The method of claim 1, wherein the subject's memory is improved.

5. A method of enhancing the metabolism of a subject, the method comprising administering Pla2g2f to the subject.

6. The method of any one of claims 1-5, wherein Pla2g2f is administered to a cell or a tissue in the subject.

7. The method of any one of claims 1-5, wherein Pla2g2f is administered systemically.

8. The method of claim 5, wherein enhancing the metabolism is exhibited by:
(a) increased locomotor activity;
(b) decreased body weight;
(c) decreased food consumption; and/or
(d) increased energy expenditure,
relative to a control.

9. The method of claim 8, wherein:
(a) the locomotor activity and/or energy expenditure is increased by about 5%, 6%, 7%, 8%, 9%, 10%, 11%, 12%, 13%, 14%, 15%, 20%, 25%, 30%, 35%, 40%, 45%, 50%, 60%, 70%, 80%, 90%, or 100%; and/or
(b) the subject's body weight and/or food consumption is decreased by 5%, 6%, 7%, 8%, 9%, 10%, 11%, 12%, 13%, 14%, 15%, 20%, 25%, 30%, 35%, 40%, 45%, 50%, 60%, 70%, 80%, 90%, or 100%,
relative to a control.

10. The method of claim 8 or 9, wherein the control is the subject prior to the administering of Pla2g2f.

11. A method of enhancing cognitive function in a subject, the method comprising increasing expression of Pla2g2f in a cell of the subject.

12. A method of delaying or reducing cognitive decline in a subject, the method comprising increasing expression of Pla2g2f in a cell of the subject.

13. A method of improving memory in a subject, the method comprising increasing expression of Pla2g2f in a cell of the subject.

14. A method of enhancing the metabolism of a subject, the method comprising increasing expression of Pla2g2f in a cell of the subject.

15. The method of any one of claims 1, 3, or 12, wherein cognitive decline in aging is delayed or reduced in the subject.

16. A method of treating a neurodegenerative disorder in a subject, the method comprising increasing expression of Pla2g2f in a cell of the subject or administering Pla2g2f to the subject.

17. The method of any one of claims 1-16, wherein administration of Pla2g2f or increasing expression of Pla2g2f decreases neuroinflammation in the subject.

18. The method of any one of claims 6 or 11-17, wherein the cell is a neuron.

19. The method of claim 18, wherein the neuron is in the central nervous system of the subject.

20. The method of claim 19, wherein the neuron is in the brain of the subject.

21. The method of any one of claims 18-20, wherein the neuron is a dentate granule cell.

22. The method of any one of claims 18-20, wherein the neuron is a neural stem cell.

23. The method of any one of claims 6 or 11-17, wherein the cell is a glial cell.

24. The method of claim 23, wherein the glial cell is in the central nervous system.

25. The method of any one of claims 1-24, wherein an expression vector encoding Pla2g2f is administered to the subject.

26. The method of claim 25, wherein the expression vector is targeted to the central nervous system.

27. The method of claim 25 or 26, wherein the expression vector is a viral vector.

28. The method of claim 27, wherein the viral vector is an adeno-associated virus vector (AAV).

29. The method of claim 27, wherein the viral vector is a lentiviral vector.

30. The method of any one of claims 1-24, wherein a nanoparticle comprising Pla2g2f or a nucleic acid sequence encoding Pla2g2f is administered to the subject.

31. The method of claim 30, wherein the nanoparticle is a lipid nanoparticle.

32. The method of any one of claims 1-24, wherein an exosome comprising Pla2g2f or a sequence encoding Pla2g2f is administered to the subject.

33. The method of any one of claims 1-32, wherein the subject has been diagnosed with memory loss.

34. The method of claim 33, wherein the memory loss is due to a neurodegenerative disorder, trauma, or aging.

35. The method of any one of claims 1-34, wherein the subject has been diagnosed with a neurodegenerative disorder.

36. The method of claim 35, wherein the neurodegenerative disorder is Alzheimer's disease, frontotemporal dementia, Parkinson's disease, or Amyotrophic Lateral Sclerosis (ALS).

37. A vector comprising a nucleic acid sequence encoding Pla2g2f.

38. The vector of claim 37, wherein the vector is a viral vector.

39. The vector of claim 38, wherein the viral vector is an AAV or lentiviral vector.

40. An exosome comprising Pla2g2f or a nucleic acid sequence encoding Pla2g2f.

41. A nanoparticle comprising Pla2g2f or a nucleic acid sequence encoding Pla2g2f.

42. The nanoparticle of claim 41, wherein the nanoparticle is a lipid nanoparticle.

43. A kit for enhancing cognitive function, improving memory, delaying or reducing cognitive decline, treating a neurodegenerative disorder, or enhancing the metabolism in a subject, the kit comprising a vector comprising a nucleic acid sequence encoding Pla2g2f and instructions for administration of the vector.

44. The kit of claim 43, wherein the vector is a viral vector.

45. The kit of claim 44, wherein the viral vector is an AAV or lentiviral vector.

46. The kit of claim 43, wherein cognitive decline is delayed or reduced in aging.

47. The method of any one of claims 1-36, wherein the subject is a human.


SEQUENCE LISTING (4 sequences)

SEQ ID NO: 1 - DNA, 633 bp, Mus musculus
  source 1..633: mol_type = unassigned DNA
atggcagatggggcacaggccaaccccaaagggttcaggaagaaggcgctggttaaacactccactggacggaagagcccaagcctcagggcctctccctctaaaacctccaggtccagcctgggtatgaagaaattctttgccatcgcagtcctggccggcagtgtggtaaccacggcccacagcagcctgctgaacctgaagtccatggtggaggccatcacacacagaaactccatcctgtcctttgtgggctacggctgctactgcgggctggggggacgcggccatcccatggatgaggtagactggtgctgccatgcccacgactgttgctatgagaagctctttgagcagggctgccgcccctacgtggaccactatgaccacaggatcgagaatggcaccatgattgtctgcactgagctcaatgagacggagtgtgacaagcagacatgcgagtgtgacaagagcctgactctgtgcctcaaggatcacccatacaggaacaagtaccgaggctacttcaacgtctactgccagggccccacacccaactgcagcatctatgacccgtacccagaggaagtcacctgtgggcatggcctccctgcgacccctgtctcaacctag

SEQ ID NO: 2 - DNA, 636 bp, Homo sapiens
  source 1..636: mol_type = unassigned DNA
atggcagatggggcaaaggccaaccccaaagggttcaaaaagaaggtgctggatagatgcttctctgggtggaggggcccacgcttcggggcctcctgtccttcaagaacctccaggtctagcctgggtatgaagaagttcttcaccgtggccatccttgctggcagcgttctgtccacagctcacggcagcctgctcaacctgaaggccatggtggaggccgtcacagggaggagcgccatcctgtccttcgtgggctacggttgctactgtgggctggggggccgtggccagcccaaggatgaggtggactggtgctgccacgcccacgactgctgctaccaggaactctttgaccaaggctgtcacccctatgtggaccactatgatcacaccatcgagaacaacactgagatagtctgcagtgacctcaacaagacagagtgtgacaagcagacatgcatgtgtgacaagaacatggttctgtgcctcatgaaccagacgtaccgagaggagtaccgtggcttcctcaatgtctactgccagggccccacgcccaactgcagcatctatgaaccgccccctgaggaggtcacctgcagtcaccaatccccagcgccccccgcccctccctag

SEQ ID NO: 3 - protein, 210 aa, synthetic construct
MADGAQANPKGFRKKALVKHSTGRKSPSLRASPSKTSRSSLGMKKFFAIAVLAGSVVTTAHSSLLNLKSMVEAITHRNSILSFVGYGCYCGLGGRGHPMDEVDWCCHAHDCCYEKLFEQGCRPYVDHYDHRIENGTMIVCTELNETECDKQTCECDKSLTLCLKDHPYRNKYRGYFNVYCQGPTPNCSIYDPYPEEVTCGHGLPATPVST

SEQ ID NO: 4 - protein, 211 aa, Mus musculus
MADGAKANPKGFKKKVLDRCFSGWRGPRFGASCPSRTSRSSLGMKKFFTVAILAGSVLSTAHGSLLNLKAMVEAVTGRSAILSFVGYGCYCGLGGRGQPKDEVDWCCHAHDCCYQELFDQGCHPYVDHYDHTIENNTEIVCSDLNKTECDKQTCMCDKNMVLCLMNQTYREEYRGFLNVYCQGPTPNCSIYEPPPEEVTCSHQSPAPPAPP